(19) United States
(12) Patent Application Publication
(10) Pub. No.: US 2025/0344934 A1
(43) Pub. Date: Nov. 13, 2025

(54) MEDICAL SYSTEM, CONTROL SYSTEM, AND WATER SUPPLY CONTROL METHOD

(71) Applicant: OLYMPUS CORPORATION, Tokyo (JP)

(72) Inventors: Naohiro TAKAZAWA, Tokyo (JP); Hidetoshi NISHIMURA, Tokyo (JP); Dai ITO, Tokyo (JP)

(73) Assignee: OLYMPUS CORPORATION, Tokyo (JP)

(21) Appl. No.: 18/960,364

(22) Filed: Nov. 26, 2024

Related U.S. Application Data
(60) Provisional application No. 63/645,375, filed on May 10, 2024.

(51) Int. Cl. A61B 1/00; A61B 1/12

(52) U.S. Cl. CPC A61B 1/00006; A61B 1/000096; A61B 1/126; A61B 1/127

(57) ABSTRACT

A medical system includes an endoscope configured to be electrically driven to move and to capture an endoscopic image, a treatment tool configured to be electrically driven to move, a processor configured to perform autonomous control of electrically-driven motions of the endoscope and the treatment tool and to control a water supply motion, and a memory configured to store a trained model trained so as to detect the treatment tool from the endoscopic image showing the treatment tool. The processor inputs the endoscopic image to the trained model to allow the trained model to detect the treatment tool from the endoscopic image, and acquires, from the trained model, a confidence level as to whether a detected target is the treatment tool. When the confidence level is equal to or smaller than a predetermined threshold, the processor executes the water supply motion of washing an objective lens of the endoscope.

### CROSS REFERENCE TO RELATED APPLICATIONS

This application is based upon and claims the benefit of priority to U.S. Provisional Patent Application No. 63/645,375 filed on May 10, 2024, the entire contents of which are incorporated herein by reference.

### BACKGROUND

During endoscope manipulation, secretion such as mucus or scattered tissue may contaminate or fog an object lens, and such contamination or the like of the object lens frequently deteriorates visibility of the endoscope. An operator manually conducts a water supply operation for an object lens when determining that the visibility is deteriorated by contamination or the like on the objective lens. In addition, when determining that the water supply operation is unable to clean the objective lens, the operator removes the contamination or the like on the objective lens, for example, by the following actions. The operator removes the contamination or the like on the objective lens, for example, by removing the endoscope from a patient's body and wiping the lens with a cleaning tool. Alternatively, the operator removes the contamination or the like on the objective lens by immersing the endoscope in a puddle in a lumen that is produced by the water supply operation.

Japanese Unexamined Patent Application Publication No. 2011-36582 discloses a method that detects contamination on an observation field surface in an endoscope. This method acquires a plurality of images captured at predetermined time intervals, divides each of the images into a plurality of regions, compares image information of the images for each of the regions to calculate a difference, counts the number of regions with no difference, and determines that the observation field surface is contaminated if the counted number of regions is equal to or larger than a threshold.

### SUMMARY

According to one aspect of the invention, there is provided a medical system comprising:
an endoscope configured to be electrically driven to move, and to capture an endoscopic image;
a treatment tool configured to be electrically driven to move;
a processor configured to perform autonomous control of electrically-driven motions of the endoscope and the treatment tool and to control a water supply motion; and
a memory configured to store a trained model trained so as to detect the treatment tool from the endoscopic image showing the treatment tool, wherein
the processor inputs the endoscopic image to the trained model to allow the trained model to detect the treatment tool from the endoscopic image, and acquires, from the trained model, a confidence level as to whether a detected target is the treatment tool, and
when the confidence level is equal to or smaller than a predetermined threshold, the processor executes the water supply motion of washing an objective lens of the endoscope.

According to one aspect of the invention, there is provided a control system comprising:
a processor configured to perform autonomous control of electrically-driven motions of an endoscope and a treatment tool and to control a water supply motion, the endoscope being configured to be electrically driven to move and to capture an endoscopic image, the treatment tool being configured to be electrically driven to move; and
a memory configured to store a trained model trained so as to detect the treatment tool from the endoscopic image showing the treatment tool, wherein
the processor inputs the endoscopic image to the trained model to allow the trained model to detect the treatment tool from the endoscopic image, and acquires, from the trained model, a confidence level as to whether a detected target is the treatment tool, and
when the confidence level is equal to or smaller than a predetermined threshold, the processor executes the water supply motion of washing an objective lens of the endoscope.

According to one aspect of the invention, there is provided a water supply control method comprising:
autonomously controlling electrically-driven motions of an endoscope and a treatment tool, the endoscope being configured to be electrically driven to move and to capture an endoscopic image, the treatment tool being configured to be electrically driven to move;
inputting the endoscopic image to a trained model trained so as to detect the treatment tool from the endoscopic image showing the treatment tool, and allowing the trained model to detect the treatment tool from the endoscopic image;
acquiring, from the trained model, a confidence level as to whether a target detected by the trained model is the treatment tool; and
when the confidence level is equal to or smaller than a predetermined threshold, executing the water supply motion of washing an objective lens of the endoscope.

### BRIEF DESCRIPTION OF THE DRAWINGS

FIG. 1 is a diagram illustrating a configuration example of a medical system.

FIG. 2 is a diagram illustrating an example of image recognition performed by a processor using a trained model when the trained model is object detection AI.

FIG. 3 is a diagram illustrating an example of image recognition performed by the processor using a trained model when the trained model is segmentation AI.

FIG. 4 is a diagram illustrating an example of image recognition performed by the processor using a trained model when the trained model is key point detection AI.

FIG. 5 is a diagram schematically illustrating an endoscopic image where lens fogging occurs.

FIG. 6 is a diagram schematically illustrating an endoscopic image where lens contamination occurs.

FIG. 7 is a graph schematically illustrating change in confidence level over time in a case where lens contamination and fogging does not occur.

FIG. 8 is a graph schematically illustrating change in confidence level over time in a case where lens contamination or fogging occurs.

FIG. 9 is a graph illustrating a method of determining whether lens contamination or fogging occurs.

FIG. 10 is a diagram illustrating change in endoscopic image in a case where lens contamination or fogging is eliminated.

FIG. 11 is a graph illustrating change in confidence level in a case where lens contamination and fogging is eliminated.

FIG. 12 is a graph illustrating change in confidence level in a case where lens contamination and fogging is not eliminated.

FIG. 13 is a diagram illustrating change in confidence level in key point detection.

FIG. 14 is a diagram illustrating a detailed configuration example of the medical system.

FIG. 15 is a flowchart example illustrating operation of the medical system according to first to eighth embodiments.

FIG. 16 is a flowchart example illustrating operation of the medical system 1 according to a ninth embodiment.

FIG. 17 is a diagram illustrating a configuration example of a training system.

FIG. 18 is a diagram illustrating a more detailed configuration example of the medical system.

FIG. 19 is a diagram illustrating a detailed configuration example of a water supply port and a water supply device for washing an objective lens.

FIG. 20 is a diagram illustrating a detailed configuration example of a driving device.

FIG. 21 is a diagram illustrating another configuration example of an endoscope distal end area.

FIG. 22 is a diagram illustrating yet another configuration example of the endoscope distal end area.

FIG. 23 is a diagram illustrating yet another configuration example of the endoscope distal end area.

FIG. 24 is a diagram illustrating a detailed configuration example of a second treatment tool driving device.

FIG. 25 is a diagram illustrating a detailed configuration example of a third treatment tool which is a high-frequency knife, and a configuration for cleaning the third treatment tool.

### DESCRIPTION OF EXEMPLARY EMBODIMENTS

The following disclosure provides many different embodiments, or examples, for implementing different features of the provided subject matter. These are, of course, merely examples and are not intended to be limiting. In addition, the disclosure may repeat reference numerals and/or letters in the various examples. This repetition is for the purpose of simplicity and clarity and does not in itself dictate a relationship between the various embodiments and/or configurations discussed. Further, when a first element is described as being “connected” or “coupled” to a second element, such description includes embodiments in which the first and second elements are directly connected or coupled to each other, and also includes embodiments in which the first and second elements are indirectly connected or coupled to each other with one or more other intervening elements in between.

### 1. Configuration Example

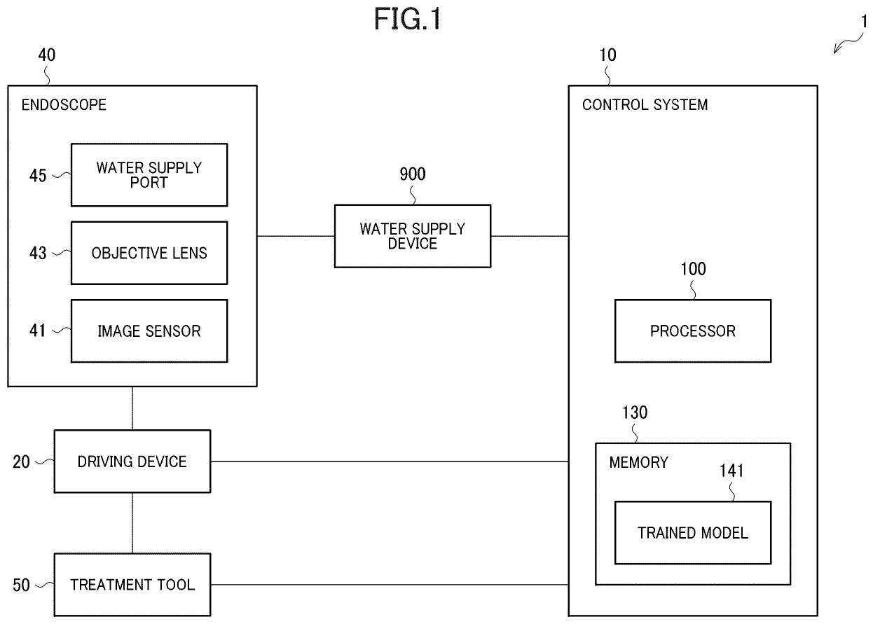

FIG. 1 illustrates a configuration example of a medical system 1. The medical system 1 may be a robot system that autonomously controls a flexible endoscope that captures an image of a body cavity such as a digestive tract. However, the medical system 1 may be a surgical system such as a surgical robot using a rigid endoscope. The medical system 1 includes a control system 10, an endoscope 40, a treatment tool 50, a driving device 20, and a water supply device 900.

The endoscope 40 and the treatment tool 50 are electrically driven, that is, moved by an electric actuator using a motor, a piezoelectric element, or the like. The driving device 20 includes an electric actuator and a circuit that controls the electric actuator, and electrically drives motions of the endoscope 40 and the treatment tool 50. The electrically driving includes electrically driving by autonomous control and electrically driving by manual operation. The autonomous control means that the medical system 1 autonomously controls the motions of the endoscope 40 and the treatment tool 50 using an endoscopic image, a sensor signal, or the like, without human intervention. The manual operation means that a human performs an operation input using a controller or the like, and the medical system 1 in turn controls the motions of the endoscope 40 and the treatment tool 50 based on the operation input.

Although the present embodiment assumes the electrically driving by autonomous control, one or more of the motions of the endoscope 40 and the treatment tool 50 may be manually operated. The motion of the endoscope 40 includes advancing and retracting, roll rotation, and angle motion. All of these motions may be autonomously controlled or only one or more of the motions may be autonomously controlled. When a plurality of treatment tools are used as the treatment tool 50, all of the treatment tools may be autonomously controlled or only one or more of the treatment tools may be autonomously controlled. Alternatively, all or one or more of the motions of the endoscope 40 and the treatment tool 50 may be switchable between autonomous control and manual operation. The motions of the endoscope 40 and the treatment tool 50 may include a non-electrically-driven motion, that is, a motion made by force applied by a human and physically transmitted without using an electric actuator. In other words, one or more of advancing and retracting, roll rotation, and angle motion of the endoscope 40 may be non-electrically driven, or the motions of one or more of a plurality of treatment tools may be non-electrically driven.

The endoscope 40 has a flexible insertion portion to be inserted into a body cavity such as a digestive tract and captures an image of the body cavity with an imaging section at a distal end of the insertion portion. The endoscope 40 may be a rigid endoscope for use in surgical operations as described above. The imaging section includes an objective lens 43 at a distal end of the endoscope 40 to form an image of a subject, and an image sensor 41 that captures the image of the subject formed by the objective lens 43. The endoscope 40 includes a water supply port 45 to supply water into the body.

The water supply device 900 includes a water supply pump and feeds a liquid to the water supply port 45 to allow the water supply port 45 to discharge the liquid. The water supply port 45 is provided at the distal end of the endoscope 40 so that a liquid is discharged toward the objective lens 43. In other words, the water supply port 45 discharges a liquid, whereby the liquid cleans an objective surface of the objective lens 43 and the liquid is fed into the body cavity. The liquid is, for example, physiological saline. The liquid is not limited to this and may be any liquid that is used in endoscope manipulation. The water supply port 45 may also serve as an air feed port or a suction port. The water supply device 900 may further include a mechanism for feeding air to the water supply port 45 serving as the air feed port, or a mechanism for suctioning gas or liquid from the water supply port 45 serving as the suction port.

The treatment tool 50 is a tool that is inserted together with the endoscope 40 into the body cavity to treat a treatment target such as a lesion. The treatment tool 50 is separatable from the endoscope 40. For example, the treatment tool 50 is inserted into a forceps channel of the endoscope 40 and protrudes from a forceps opening in use. Alternatively, the treatment tool 50 may be a robot arm provided at the distal end of the endoscope 40. The treatment tool 50 is arranged such that a distal end portion of the treatment tool 50 is shown in a field of view of the endoscope 40 in manipulation. In other words, in a forward-viewing endoscope, a field of view of the imaging section is oriented forward of the endoscope, and the treatment tool 50 is provided so as to protrude from a distal end surface of the endoscope. Alternatively, the endoscope 40 may be a side-viewing endoscope, in which the field of view of the imaging section is oriented sideways at the distal end of the endoscope, and the treatment tool 50 may be provided so as to protrude from a side surface in the vicinity of the distal end of the endoscope.

The treatment tool 50 is, for example, grasping forceps, spatula, knife, energy device, injection needle, lithotripsy basket, or catheter. The energy device is a device that applies electrical or ultrasonic energy to tissue to incise, resect, or clot the tissue. An example of the energy device is a high-frequency knife, which incises tissue in contact with the distal end of the treatment tool by feeding high-frequency current between an electrode at the distal end of the treatment tool and an electrode outside the body. The energy device having one such electrode is called a monopolar device. Alternatively, the energy device may be a bipolar device that feeds high-frequency current between two electrodes in the shape of forceps at the distal end of the treatment tool. Alternatively, the energy device may be a high-frequency snare that feeds high-frequency current to a metal ring to resect a polyp or the like. One or more treatment tools 50 may be used. When a plurality of treatment tools are used, the treatment tools are configured to be electrically driven to move independently of each other. The electrically-driven motion of the treatment tool includes advancing and retracting, roll rotation, angle motion, or opening and closing of the forceps.

The control system 10 includes a processor 100 and a memory 130. The processor 100 generates image data of an endoscopic image by performing image processing on image data captured by the endoscope 40. Hereinafter the image data of an endoscopic image is simply referred to as endoscopic image. An image processing system that generates an endoscopic image may be provided separately from the control system 10. In this case, the control system 10 receives the endoscopic image from the image processing system. The memory 130 stores a trained model 141 trained so as to detect the treatment tool from the endoscopic image. The trained model 141 is a machine learning model including a neural network and detects the treatment tool from the endoscopic image using an image recognition method such as object detection, segmentation, or key point detection. The processor 100 inputs the endoscopic image to the trained model 141 to detect the treatment tool from the endoscopic image and autonomously controls each section of the medical system 1 using the detection result.

Specifically, the trained model 141 outputs a detection result of the treatment tool as well as a confidence level that a detected object is the treatment tool. The confidence level is represented by a real number, for example, from 0 to 1. With a confidence level closer to 1, the trained model 141 determines that a detected object is the treatment tool. The processor 100 compares the confidence level with a predetermined threshold. When the confidence level is equal to or smaller than the predetermined threshold, the processor 100 controls the water supply device 900 so that water is supplied from the water supply port 45. As a result, the objective surface of the objective lens 43 is cleaned. In other words, when the confidence level is equal to or smaller than the predetermined threshold, the processor 100 determines that the accuracy of image recognition is reduced due to contamination or fogging of the objective lens 43, and improves visibility by cleaning the objective lens 43.

In addition, the processor 100 autonomously controls the electrically-driven motions of the endoscope 40 and the treatment tool 50 by controlling the driving device 20 based on the detection result of the treatment tool by the trained model 141. In other words, the processor 100 determines one or more or all of the treatment tool's position, direction, shape, and state of contact with tissue from the detection result of the treatment tool, determines a next motion in manipulation based on the determination result, and controls the endoscope 40 and the treatment tool 50 so that the next motion is performed. The memory 130 may further include a scene detection trained model. The scene detection trained model is trained so as to determine a manipulation scene from the endoscopic image, the detection result of the treatment tool by the trained model 141, or both of the endoscopic image and the detection result of the treatment tool. The manipulation scene is determined from the position, shape, or boundary of tissue such as a lesion, or a positional relation between the tissue and the treatment tool, in addition to the position and the like of the treatment tool. Further, when the endoscope 40 or the treatment tool 50 has a sensor such as a force sensor or a shape detection sensor, the scene detection trained model may be trained so as to determine a manipulation scene additionally using a sensor output. The processor 100 may determine a next motion in manipulation, based on a scene detection result, or a scene detection result and a detection result of the treatment tool by the trained model 141, and may control the endoscope 40 and the treatment tool 50 so that the next motion is performed.

Further, when the motions of the endoscope 40 and the treatment tool 50 are manually operated, the processor 100 may control the motion of the endoscope 40 or the treatment tool 50 by controlling the driving device 20 in accordance with an operation input from a not-illustrated controller.

As described in the background, contamination or fogging of the objective lens may deteriorate the visibility of the endoscope in endoscope manipulation. In a case where the endoscope is autonomously controlled and robotized in a medical system including the endoscope, the medical system needs to autonomously recognize that the visibility is deteriorated due to contamination or the like of the objective lens. If the medical system continues autonomous control of manipulation using the endoscopic image while the visibility is deteriorated with the contaminated objective lens, the medical system may be unable to execute appropriate manipulation because the accuracy of image recognition is reduced.

In the present embodiment, the medical system 1 includes the endoscope 40 that is electrically driven to move and captures an endoscopic image, and the treatment tool 50 that is electrically driven to move. The medical system 1 further includes the processor 100 and the memory 130 that stores the trained model 141. The processor 100 autonomously controls the electrically-driven motions of the endoscope 40 and the treatment tool 50 and controls a water supply motion. The trained model 141 is a model trained so as to detect the treatment tool 50 from the endoscopic image showing the treatment tool 50. The processor 100 inputs the endoscopic image to the trained model 141 to allow the trained model 141 to detect the treatment tool 50 from the endoscopic image and acquires, from the trained model 141, the confidence level as to whether a detected target is the treatment tool 50. When the confidence level is equal to or smaller than a predetermined threshold, the processor 100 executes a water supply motion of washing the objective lens 43 of the endoscope 40.

According to the present embodiment, the presence or absence of contamination or fogging of the objective lens is determined using the confidence level output by artificial intelligence (hereinafter abbreviated as AI) that detects the treatment tool from an image, and when it is determined that contamination or fogging of the objective lens is present, the objective lens is cleaned. In the present embodiment, AI that directly detects contamination is not used but AI that detects the treatment tool from an image is used. The treatment tool is disposed near a lesion desired to be observed during surgery. Thus, when the confidence level is high and an image of the treatment tool is clearly recognized, an image of the lesion desired to be observed during surgery is also considered as being clearly captured. Further, the treatment tool is autonomously controlled using the result of image recognition of the treatment tool. Thus, when the confidence level is high and an image of the treatment tool is clearly recognized, autonomous control can also be performed appropriately. Further, frequent cleaning is bothersome, so cleaning is performed only when the treatment tool area is less visible. In this way, contamination or fogging of the objective lens is determined using AI that detects the treatment tool from an image, whereby the objective lens is efficiently cleaned to ensure a field of view for AI image recognition.

According to Japanese Unexamined Patent Application Publication No. 2011-36582 described above, it is determined that there is contamination if the count of regions with no difference as a result of comparison (contaminated regions) is equal to or larger than a threshold. In this respect, the present embodiment has the following advantages. (i) The confidence level of the treatment tool is used to detect contamination that overlaps the treatment tool in the field of view. In other words, in a region that does not overlap the treatment tool in the field of view, no contamination is recognized, and therefore water is not supplied frequently more than necessarily and smooth operation is possible. When an operator performs manipulation, the operator pays attention to an area around the treatment tool. This applies to manipulation by autonomous control. (ii) Since the treatment tool is kept at a certain distance from the endoscope lens to some extent, detecting contamination from a treatment tool image has a higher accuracy of contamination detection. A site of operation with the endoscope is frequency changed, so how the site of operation is seen is unstable: for example, the objective lens comes into contact with tissue to cause a reddish image. The reddish image means that most of the image appears red due to the tissue that the lens comes into contact with. Thus, the accuracy of contamination detection may be less ensured when contamination of the objective lens is detected from a region showing the site of operation in the image.

Another possible method for detecting contamination of the objective lens is a technique that “implements contamination detection AI trained with contamination patterns”. In this case, AI specialized in contamination detection is implemented. In this respect, according to the present embodiment, the confidence level already obtained by recognition AI or control AI, which is a main function of a robot endoscope, can be reused. This configuration eliminates the need for additionally implementing an AI model (deep learning model) dedicated to contamination detection and therefore has the advantage of suppressing additional calculation resources (for example, GPU), calculation time, and training cost.

The present embodiment may be implemented as a water supply control method. The water supply control method includes a step of autonomously controlling the electrically-driven motions of the endoscope 40 and the treatment tool 50. The endoscope 40 is electrically driven to move and captures an endoscopic image. The treatment tool 50 is electrically driven to move. The water supply control method includes a step of inputting the endoscopic image to the trained model 141 trained so as to detect the treatment tool 50 from the endoscopic image showing the treatment tool 50, and allowing the trained model 141 to detect the treatment tool 50 from the endoscopic image. The water supply control method includes a step of acquiring, from the trained model 141, a confidence level as to whether a target detected by the trained model 141 is the treatment tool 50. The water supply control method includes a step of, when the confidence level is equal to or smaller than a predetermined threshold, executing the water supply motion of washing the objective lens 43 of the endoscope 40.

The water supply control method described above may be implemented as a method of operating the medical system 1 or the control system 10 including the processor 100 and the memory 130. In this case, the processor 100 executes each of the above steps.

The processor 100, the memory 130, and the trained model 141 of the control system 10 may be configured as follows.

The processor 100 includes hardware. The processor 100 is, for example, a central processing unit (CPU), a graphics processing unit (GPU), a microcomputer, a digital signal processor (DSP), or the like. Alternatively, the processor 100 may be an application specific integrated circuit (ASIC), a field programmable gate array (FPGA), or the like. The processor 100 may include one or more of CPU, GPU, microcomputer, DSP, ASIC, FPGA, and the like. The memory 130 is, for example, a semiconductor memory which is a volatile memory or a nonvolatile memory. Alternatively, the memory 130 may be a magnetic storage device such as a hard disk device, an optical storage device such as an optical disk device, or the like.

The memory 130 stores a program that describes various processing contents such as image processing, autonomous control of the endoscope and the treatment tool, and water supply control described above. The processor 100 executes the program to execute various processing. The memory 130 stores the trained model 141 for detecting the treatment tool or the trained model for detecting a manipulation scene. These trained models may include, for example, a program that describes AI algorithms and data used in the program. For example, the trained model may include a neural network such as convolutional neural network (CNN). In this case, the trained model includes a program that describes an algorithm of a neural network, and weight parameters and biases between nodes of the neural network. The neural network includes an input layer that receives image data, an intermediate layer that performs computation processing on data input through the input layer, and an output layer that outputs a recognition result data based on a computation result output from the intermediate layer.

The program may be stored in a non-transitory information storage medium which is a computer-readable medium. The information storage medium is, for example, an optical disk, a memory card, a hard disk drive, or a semiconductor memory. The semiconductor memory is, for example, a ROM or a nonvolatile memory. The processor 100 loads a program stored in the information storage medium into the memory 130 and performs various processing based on the program.

The control system 10 is configured with an information processing device such as a personal computer, a server, or a processing device dedicated to a medical system. In this case, a processor and a memory included in the information processing device correspond to the processor 100 and the memory 130 of the control system 10. Alternatively, the control system 10 may be a cloud system to which a plurality of information processing devices are connected via a network. In this case, a processor and a memory included in the information processing device included in the cloud system may correspond to the processor 100 and the memory 130 of the control system 10.

### 2. Detailed Configuration Example

An embodiment in a case where a treatment is performed using grasping forceps and a high-frequency knife will be described below. Here, the treatment is an operation executed on a patient during a case. However, the medical system 1 may have a variety of configurations as described above. For example, one treatment tool or two or more treatment tools may be used in manipulation.

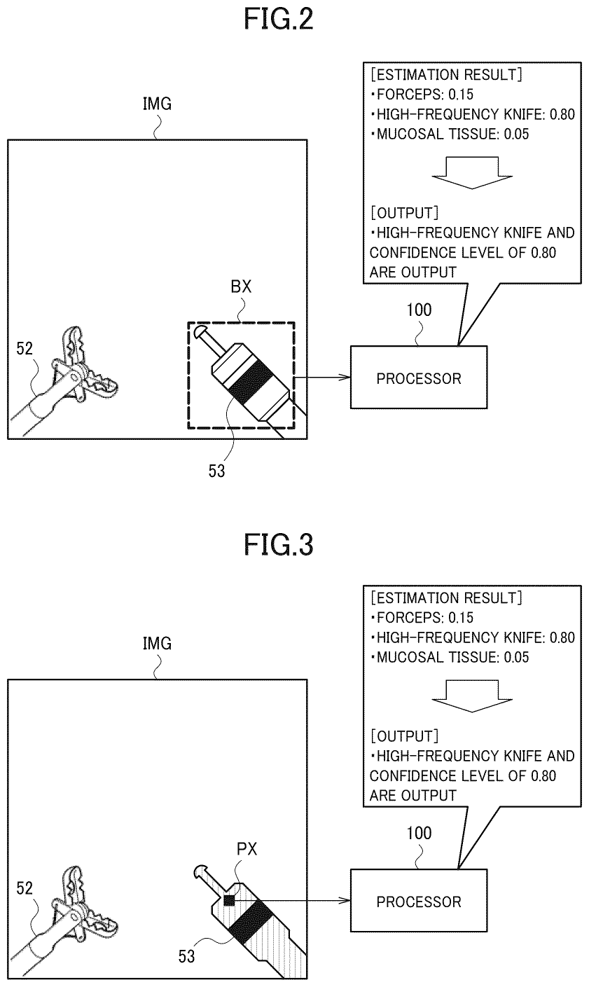

FIG. 2 to FIG. 12 are illustrations of a first embodiment.

(1) The processor 100 detects a treatment tool from an endoscopic image through image recognition using the trained model 141. The trained model 141 is AI for object detection, segmentation, key point detection, or the like. The endoscopic image is, for example, each of frame images of moving images. Alternatively, the endoscopic image may be, for example, each of still images successively captured at certain intervals.

FIG. 2 illustrates an example of image recognition performed by the processor 100 using the trained model 141 when the trained model 141 is object detection AI. Grasping forceps 52 and a high-frequency knife 53 are shown in an endoscopic image IMG. The processor 100 calculates a probability that a certain target is each of classes. The classes are, for example, the treatment tool and tissue. Types of treatment tools may be divided into classes, and types of tissue may be divided into classes. The processor 100 estimates that a class with the highest probability is the class of the target and outputs an estimation result. The probability of the class output as the estimation result is called confidence level, confidence coefficient, or confidence. Hereinafter “confidence level” is used.

In the example in FIG. 2, the classes are forceps, high-frequency knife, and mucosal tissue. The processor 100 sets the high-frequency knife 53 as a target and estimates that the confidence levels of the forceps, the high-frequency knife, and the mucosal tissue are 0.15, 0.80, and 0.05, respectively. In this case, the processor 100 outputs that the target is the high-frequency knife and its confidence level is 0.80. The processor 100 determines whether lens cleaning is necessary using the confidence level of 0.80. In a case where an estimation result is reflected in a display image, the processor 100 superimposes a boundary box BX on a region where the high-frequency knife is detected in the endoscopic image IMG, and displays the endoscopic image IMG after superimposition on a display. The processor 100 may perform estimation for the grasping forceps 52 as a target, and output an estimation result and a confidence level of the grasping forceps.

FIG. 3 illustrates an example of image recognition performed by the processor 100 using the trained model 141 when the trained model 141 is segmentation AI. The processor 100 calculates a probability of each class for each pixel of the endoscopic image IMG. The processor 100 estimates that a class with the highest probability in each pixel is the class of the pixel and outputs an estimation result.

In the example in FIG. 3, the processor 100 sets a pixel PX as a target and estimates that the confidence levels of the forceps, the high-frequency knife, and the mucosal tissue are 0.15, 0.80, and 0.05, respectively. In this case, the processor 100 outputs that the pixel PX is the high-frequency knife and its confidence level is 0.80. The processor 100 determines whether lens cleaning is necessary using the confidence level of 0.80. The processor 100 performs estimation for each pixel. Thus, usually, there are a plurality of pixels determined as being the high-frequency knife. The processor 100 may determine whether lens cleaning is necessary, based on the confidence levels of the high-frequency knife in a plurality of pixels determined as being the high-frequency knife. In a case where an estimation result is reflected in a display image, the processor 100 highlights the pixels where the high-frequency knife is detected in the endoscopic image IMG, and displays the endoscopic image IMG after highlighting on a display. In FIG. 3, the highlighted section is hatched. The processor 100 may perform estimation for the grasping forceps 52 as a target, and output an estimation result and a confidence level of the grasping forceps.

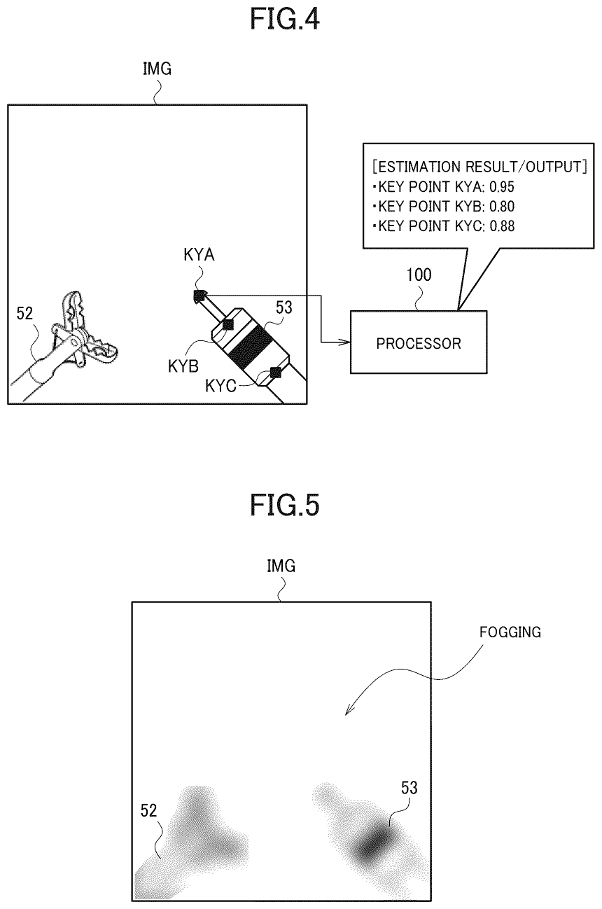

FIG. 4 illustrates an example of image recognition performed by the processor 100 using the trained model 141 when the trained model 141 is key point detection AI. The processor 100 calculates a map of confidence level of each key point for a certain target. The key point is a position of an element or a characteristic point of the treatment tool, for example, a position of the distal end of the treatment tool, a joint position, a position of a component, or a position of connection between components. The map of confidence level is called heat map and indicates a distribution of confidence level of the key point in an image. In other words, in the map of confidence level, the confidence level of the key point in each pixel is allocated to the pixel. The processor 100 estimates that a position with the highest confidence level for each key point is the position of the key point, and outputs an estimation result.

The high-frequency knife has a cylindrical rigid portion at a distal end of a tube, and a knife at a distal end of the rigid portion. FIG. 4 illustrates an example in which a key point KYA, a key point KYB, and a key point KYC are detected. The key point KYA is a knife distal end. The key point KYB is a connection point between the knife and the rigid portion. The key point KYC is a connection point between the rigid portion and the tube. The processor 100 calculates a heat map for each of the key points KYA, KYB, and KYC and estimates that positions with the highest confidence level in the respective heat maps are the key points KYA, KYB, and KYC. It is assumed that the confidence levels at the positions estimated as the key points KYA, KYB, and KYC are 0.95, 0.80, and 0.88, respectively. The processor 100 outputs these confidence levels as an estimation result. The processor 100 determines whether lens cleaning is necessary, based on the confidence levels of the key points KYA, KYB, and KYC. The processor 100 may use the confidence levels of all of the key points or may use the confidence levels of one or more of the key points KYA, KYB, and KYC. In a case where the estimation result is reflected in a display image, the processor 100 superimposes a point, a mark, or the like at a position where each key point is detected in the endoscopic image IMG, and displays the endoscopic image IMG after superimposition on a display.

In a case where the key point detection is used, if the confidence level of a specific key point in the treatment tool is reduced, it can be determined that contamination or fogging of the objective lens is present at the specific key point position. In this way, the key point detection can be used to narrow down the position of contamination or fogging on the objective lens to some extent.

(2) The processor 100 monitors the confidence level continuously or at any frame intervals.

FIG. 5 schematically illustrates the endoscopic image IMG where lens fogging occurs. For example, fogging occurs in the objective lens when water vapor in the body cavity or body fluid evaporated by a treatment is condensed, or when body fluid adheres to the endoscope distal end in contact with the body cavity. Since lens fogging blurs the endoscopic image, the grasping forceps 52 and the high-frequency knife 53 are shown blurred.

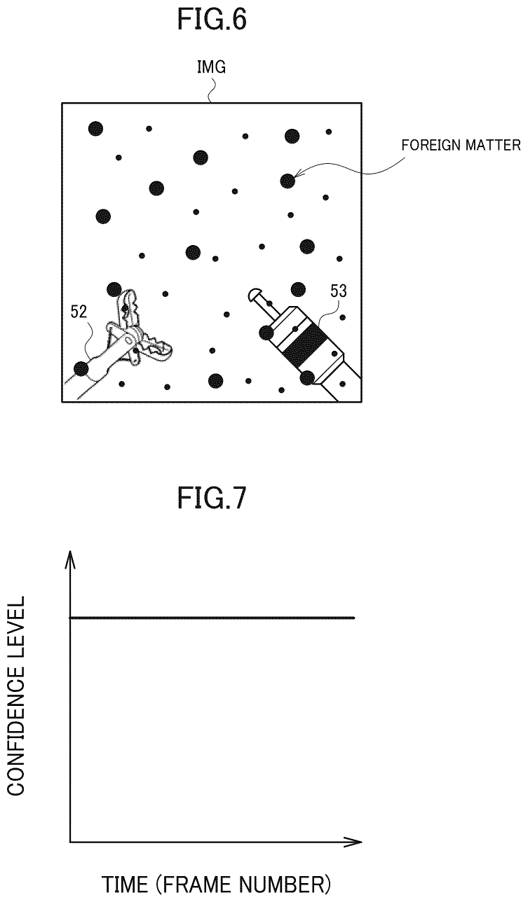

FIG. 6 schematically illustrates the endoscopic image IMG where lens contamination occurs. For example, contamination occurs in the objective lens when a tissue fragment or liquid scattered by a treatment adheres, or when a tissue fragment or body fluid adheres to the endoscope distal end in contact with the body cavity. When lens contamination occurs, foreign matter is shown in the endoscopic image, so that the grasping forceps 52 and the high-frequency knife 53 are partially interrupted by the foreign matter.

FIG. 7 schematically illustrates change in confidence level over time in a case where lens contamination and fogging does not occur. The horizontal axis represents time. Not limited to hour, minute, and second, the horizontal axis may represent any information that indicates a time series, such as frame numbers. When lens contamination and fogging does not occur, the confidence level changes little over time. FIG. 7 illustrates an example in which the confidence level is constant. However, in practice, the confidence level may vary and the confidence level transitions while keeping a high value to some extent.

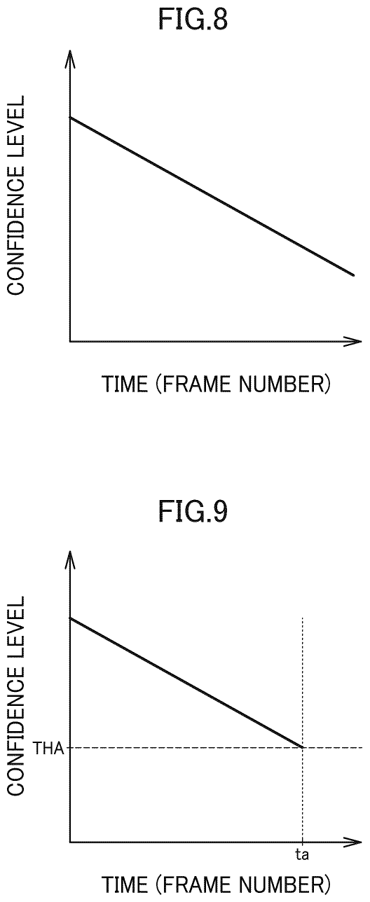

FIG. 8 schematically illustrates change in confidence level over time in a case where lens contamination or fogging occurs. When lens contamination or fogging occurs, the confidence level changes over time. For example, as the contamination or fogging increases, the confidence level gradually decreases.

The processor 100 monitors the confidence level as follows.
In a case where the object detection AI is used, the confidence level is monitored for each class.
In a case where the segmentation AI is used, the processor 100 monitors a statistical value of the confidence level for each class. The statistical value is mean, median, maximum, or the like. For example, the processor 100 uses the confidence levels of pixels determined as the high-frequency knife as a population and calculates the mean, median, maximum, or the like of the confidence level of the high-frequency knife.
In a case where the key point detection AI is used, the processor 100 monitors, for each key point, the confidence level of a detected key point position or a statistical value of the confidence level in a plurality of surrounding pixels including a key point position. The statistical value is mean, median, maximum, or the like.
Since a monitored target is the treatment tool present in the endoscopic image, only a class having a confidence level equal to or larger than a given threshold is monitored. This is because a class that does not have a confidence level equal to or larger than the given threshold, that is, a class having too small a confidence level, can be considered as not being present in the endoscopic image. Here, the given threshold may be different from the threshold used for determining whether lens cleaning is necessary.

(3) The processor 100 compares the confidence level with a predetermined threshold and, when the confidence level is equal to or smaller than the predetermined threshold, the processor 100 determines that lens contamination or fogging may occur.

When the confidence level is output in a range from 0 to 1, for example, the predetermined threshold may be set as follows. In a treatment scene in which the treatment tool incises or grasps tissue, the predetermined threshold is, for example, 0.8 or more. In a treatment scene in which the treatment tool does not incise or grasp tissue but the endoscope is moving, the predetermined threshold is, for example, 0.5 or more. In a treatment scene in which the treatment tool does not incise or grasp tissue and the endoscope is not moving, the predetermined threshold is 0.3 or more. However, these thresholds are only by way of example and any appropriate threshold can be set in accordance with a treatment scene. Alternatively, a fixed threshold may be used irrespective of a treatment scene.

FIG. 9 illustrates a method of determining whether lens contamination or fogging occurs. When the confidence level is higher than a predetermined threshold THA, the processor 100 determines that lens contamination or fogging does not occur. When the confidence level is equal to or smaller than the predetermined threshold THA, the processor 100 determines that lens contamination or fogging occurs. FIG. 9 illustrates an example in which the confidence level gradually decreases from a state higher than the predetermined threshold THA, and the confidence level reaches the predetermined threshold THA at time ta. It is not intended to preclude the processor 100 from determining that lens contamination or fogging occurs when the confidence level is initially equal to or smaller than the predetermined threshold THA.

(4) When the processor 100 determines that lens contamination or fogging may occur, the processor 100 autonomously executes a water supply motion to the objective lens 43. In other words, the processor 100 transmits a water supply instruction to the water supply device 900, and the water supply device 900 receives the water supply instruction and supplies water from the water supply port 45 to the objective lens 43.

(5) When the confidence level becomes equal to or larger than the threshold as a result of the water supply motion, the processor 100 determines that lens contamination or fogging is eliminated, and permits continuation of the treatment by the medical system 1.

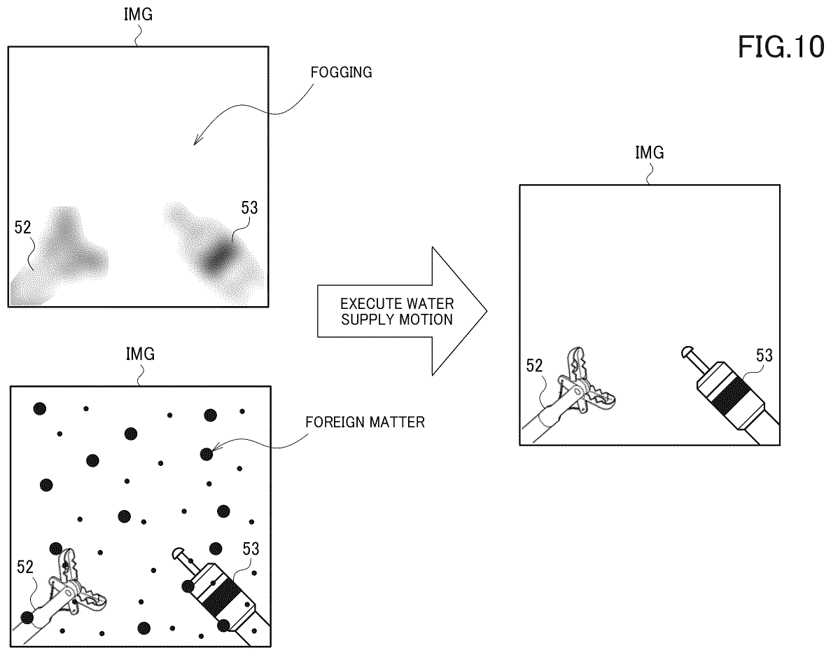

FIG. 10 illustrates change in endoscopic image IMG in a case where lens contamination or fogging is eliminated. The water supply cleans the objective lens 43, thereby eliminating contamination or fogging of the objective lens 43. As a result, the grasping forceps 52 and the high-frequency knife 53 are clearly shown in the endoscopic image IMG.

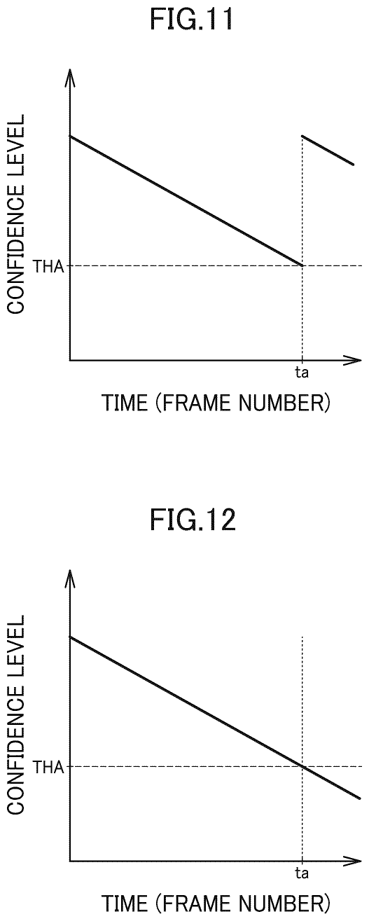

FIG. 11 illustrates change in confidence level in a case where lens contamination and fogging is eliminated. At time ta, the confidence level becomes equal to or smaller than the predetermined threshold THA. The objective lens 43 is cleaned, and the confidence level becomes higher than the predetermined threshold THA again. This means that the cleaning restores the field of view to enable image recognition AI to accurately recognize the treatment tool. The processor 100 autonomously controls the endoscope and the treatment tool based on the endoscopic image before time ta and continues autonomous control even after time ta.

(6) When the confidence level does not become equal to or larger than the threshold as a result of the water supply motion, the processor 100 determines that lens contamination or fogging is not eliminated, and does not permit continuation of the treatment by the medical system 1. In other words, the processor 100 stops the treatment.

FIG. 12 illustrates change in confidence level in a case where lens contamination and fogging is not eliminated. At time ta, the confidence level becomes equal to or smaller than the predetermined threshold THA. The objective lens 43 is cleaned, but the confidence level is kept at the predetermined threshold THA or smaller and is not restored. This means that the cleaning does not restore the field of view and image recognition AI does not accurately recognize the treatment tool. The processor 100 autonomously controls the endoscope and the treatment tool based on the endoscopic image before time ta and stops autonomous control after time ta.

When the confidence level does not become equal to or larger than the threshold as a result of the water supply motion, the processor 100 may perform the water supply motion again to clean the lens. When the confidence level does not become equal to or larger than the threshold even after repeating the lens cleaning again one or more times, the processor 100 may not permit continuation of the treatment by the medical system 1.

(7) When the confidence level does not become equal to or larger than the threshold as a result of the water supply motion, the processor 100 determines that lens contamination or fogging is not eliminated, and makes notification to a user. In other words, the processor 100 notifies the user to remove the endoscope from the patient's body and manually remove the lens contamination or fogging. The notification may be made by display on a monitor or by an alarm. The alarm may be sound, light, vibration, or the like. Both (6) and (7) may be conducted, or one of them may be conducted.

In a second embodiment, (3) in the first embodiment is changed to the following (3a).

(3a) The processor 100 compares the confidence level with the predetermined threshold and, when the confidence level is equal to or smaller than the predetermined threshold over a predetermined length of time, the processor 100 determines that lens contamination or fogging may occur. It can be said that the predetermined length of time is a predetermined number of frames. For example, when the confidence level is equal to or smaller than the predetermined threshold in all of the frames among a predetermined number of successive frames, the processor 100 determines that lens contamination or fogging may occur. The second embodiment can improve immunity to noise appearing in the confidence level.

In a third embodiment, (3) in the first embodiment is changed to the following (3b).

(3b) The processor 100 performs smoothing processing on time-series data of the confidence level and compares the confidence level subjected to the smoothing processing with the predetermined threshold. When the confidence level subjected to the smoothing processing is equal to or smaller than the predetermined threshold, the processor 100 determines that lens contamination or fogging may occur. The smoothing processing is, for example, a moving average method that averages the time-series data of the confidence level in the predetermined length of time and sequentially shifts the time period. Alternatively, the smoothing processing may be a low-pass filter, a band-pass filter, or the like. The third embodiment can further improve immunity to noise appearing in the confidence level.

In a fourth embodiment, (5) in the first embodiment is changed to the following (5a).

(5a) When the confidence level is higher than the predetermined threshold over a predetermined length of time after the water supply motion, the processor 100 determines that lens contamination or fogging is eliminated, and permits continuation of the treatment by the medical system 1. It can be said that the predetermined length of time is a predetermined number of frames. For example, when the confidence level is higher than the predetermined threshold in all of the frames among a predetermined number of successive frames after the water supply motion, the processor 100 determines that lens contamination or fogging may occur. The predetermined length in (5a) may be different from the predetermined length in (3a). The fourth embodiment can improve immunity to noise appearing in the confidence level. The fourth embodiment may be combined with the second and third embodiments.

In a fifth embodiment, (5) in the first embodiment is changed to the following (5b).

(5b) The processor 100 performs smoothing processing on time-series data of the confidence level and compares the confidence level subjected to the smoothing processing with the predetermined threshold. When the confidence level subjected to the smoothing processing is higher than the predetermined threshold after the water supply motion, the processor 100 determines that lens contamination or fogging is eliminated, and permits continuation of the treatment by the medical system 1. The smoothing processing is the moving average method, the low-pass filter, the band-pass filter, or the like. The fifth embodiment can further improve immunity to noise appearing in the confidence level. The fifth embodiment may be combined with the second and third embodiments.

In a sixth embodiment, the following (8) is added to the first embodiment.

(8) The processor 100 obtains a differential value of the confidence level as a change in confidence level, calculates how long it takes before a water supply operation needs to be performed, and allows the water supply motion to be performed at an appropriate timing in the treatment. The processor 100 estimates the time taken for the confidence level to reach the predetermined threshold, where the confidence level at present is an initial value and the confidence level changes with a differential value of the confidence level. The timing when the confidence level is estimated to reach the predetermined threshold is referred to as scheduled water supply timing. If there is an appropriate timing in the treatment before the scheduled water supply timing, that is, even when the confidence level does not reach the predetermined threshold, the processor 100 performs the water supply motion at that timing. For example, when a treatment step programmed in advance is terminated, the processor 100 executes the water supply motion at a transition timing between the terminated treatment step and the next treatment step. According to the sixth embodiment, lens cleaning is performed at an appropriate timing such as between treatment steps. This configuration does not interrupt a treatment procedure and prevents reduction in efficiency of the treatment procedure. The sixth embodiment may be combined with the second to fifth embodiments.

In a seventh embodiment, (8) in the sixth embodiment is changed to the following (8a).

(8a) The time estimation using a differential value of the confidence level is the same as in (8). The processor 100 performs scene detection from the endoscopic image or the like using a scene recognition trained model, and determines an appropriate timing for lens cleaning based on a scene detection result. When the confidence level does not reach the predetermined threshold but the processor 100 determines that there is an appropriate timing as a result of scene detection, the water supply motion is performed at that timing. For example, the processor 100 detects termination of a treatment step as a result of scene detection, and executes the water supply motion at a transition timing between the terminated treatment step and the next treatment step. The seventh embodiment can prevent reduction in efficiency of a treatment procedure more accurately. The seventh embodiment may be combined with the second to fifth embodiments.

In an eighth embodiment, the following (9) is added to the first embodiment.

(9) The processor 100 allows washing of the treatment tool before lens contamination or fogging is detected, and allows detection of only lens contamination or fogging. Specifically, the treatment tool has a water supply port for supplying water and cleaning the distal end portion of the treatment tool. The water supply device 900 has, for example, a pump that supplies water to the water supply port of the treatment tool. The processor 100 first transmits an instruction to the water supply device 900 to supply water to the treatment tool, and the water supply device 900 supplies water to the water supply port of the treatment tool. As a result, the tissue, body fluid, or the like adhering to the treatment tool is cleaned off. Subsequently, the processor 100 starts comparing the confidence level with the predetermined threshold. When the confidence level becomes equal to or smaller than the predetermined threshold, the processor 100 transmits an instruction to the water supply device 900 to supply water to the objective lens, and the water supply device 900 supplies water to the objective lens. As a result, contamination or fogging of the objective lens is cleaned off. A configuration that supplies water to the treatment tool will be described later. The eighth embodiment may be combined with the second to seventh embodiments.

In a ninth embodiment, the following (10) is added to the first embodiment.

(10) The processor 100 detects the treatment tool using the key point detection AI. In this case, when the confidence level decreases only in one or more specific key points, the processor 100 determines that the treatment tool itself is contaminated, rather than contamination or fogging of the objective lens, and permits continuation of the treatment by the medical system 1.

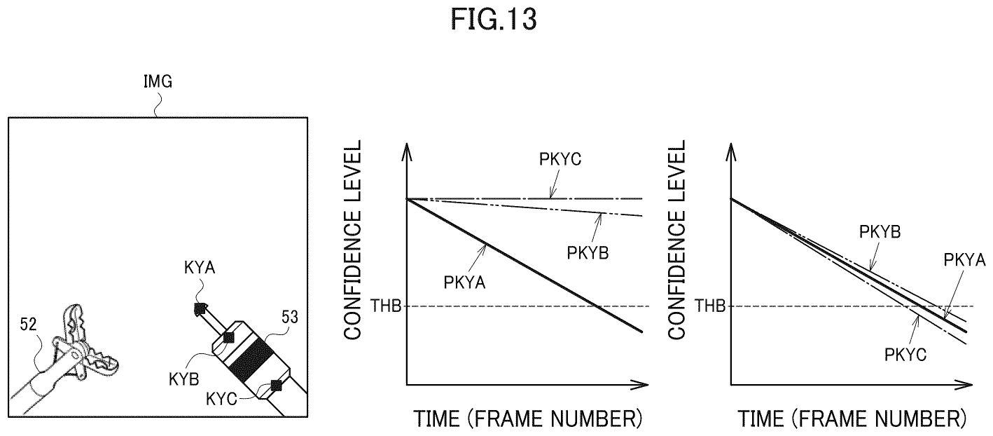

FIG. 13 illustrates an example of change in confidence level in the key point detection. As illustrated in the left diagram in FIG. 13, it is assumed that the key points KYA, KYB, and KYC of the high-frequency knife 53 are detection targets. As illustrated in the center graph in FIG. 13, it is assumed that only a confidence level PKYA of the key point KYA decreases and becomes equal to or smaller than a predetermined threshold THB, whereas confidence levels PKYB and PKYC of the key points KYB and KYC do not virtually change and do not become equal to or smaller than the threshold THB. In this case, the processor 100 continues autonomous treatment control using image recognition. When the confidence level PKYA becomes equal to or smaller than the predetermined threshold THB, the processor 100 may or may not perform the water supply motion to the objective lens, or may perform the water supply motion to the high-frequency knife 53.

As illustrated in the right graph in FIG. 13, it is assumed that the confidence levels PKYA, PKYB, and PKYC of all of the key points KYA, KYB, and KYC decrease and become equal to or smaller than the predetermined threshold THB. In this case, the processor 100 stops the autonomous treatment control using image recognition. For example, the processor 100 stops the autonomous treatment control using image recognition when the confidence levels of all of the key points become equal to or smaller than the predetermined threshold THB within a predetermined time after the confidence level of one key point becomes equal to or smaller than the predetermined threshold THB. The processor 100 may perform the water supply motion to the objective lens when the confidence levels of all of the key points become equal to or smaller than the predetermined threshold THB. In this case, the processor 100 may continue the autonomous treatment control using image recognition when there is a key point of which confidence level is higher than the predetermined threshold THB after the water supply motion. The processor may stop the autonomous treatment control using image recognition when the confidence levels of all of the key points are equal to or smaller than the predetermined threshold THB after the water supply motion.

The ninth embodiment can prevent the medical system 1 from stopping the treatment as a result of an erroneous determination of lens contamination or fogging when the confidence level decreases not due to lens contamination or fogging but due to contamination of the treatment tool. The ninth embodiment may be combined with the second to eighth embodiments.

The same treatment stop determination as described above can be conducted even when a plurality of treatment tools are detected in the object detection or the segmentation, in addition to the key point detection. For example, it is assumed that a first treatment tool and a second treatment tool are detected in the object detection or the segmentation, and a first confidence level and a second confidence level corresponding to these tools are obtained. The processor 100 continues the autonomous treatment control using image recognition when the confidence level of only the first treatment tool or the second treatment tool becomes equal to or smaller than the predetermined threshold. Further, the processor 100 may execute the water supply motion for the treatment tool of which confidence level becomes equal to or smaller than the predetermined threshold. The processor 100 stops the autonomous treatment control using image recognition when the confidence levels of both the first treatment tool and the second treatment tool become equal to or smaller than the predetermined threshold.

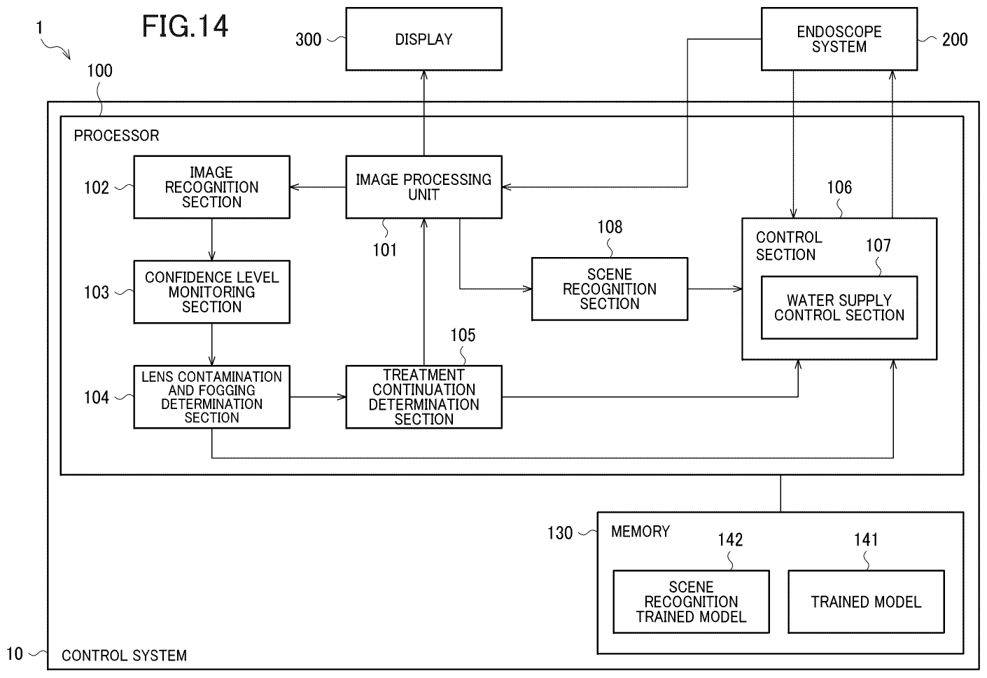

FIG. 14 illustrates a detailed configuration example of the medical system 1. The medical system 1 includes the control system 10, an endoscope system 200, and a display 300. The endoscope system 200 corresponds to the endoscope 40, the treatment tool 50, the driving device 20, and the water supply device 900 in FIG. 1. The control system 10 includes the processor 100 and the memory 130.

The processor 100 executes processing as an image processing section 101, an image recognition section 102, a confidence level monitoring section 103, a lens contamination and fogging determination section 104, a treatment continuation determination section 105, a control section 106, and a scene recognition section 108. In other words, the memory 130 stores a program that describes the contents of processing of each of the above sections, and the processor 100 executes the program to execute the processing of each section. The memory 130 stores the trained model 141 and a scene recognition trained model 142. The trained model 141 is trained so as to recognize a treatment tool and the like from an input endoscopic image. The scene recognition trained model is trained so as to recognize a treatment scene or a treatment step from an input endoscopic image.

The image processing section 101 generates image data of the endoscopic image from an image capturing signal received from the image sensor 41 of the endoscope 40. The image processing section 101 outputs the endoscopic image to the display 300, and the display 300 displays the endoscopic image. An image processing device may be provided separately from the control system 10, and a processor of the image processing device may execute the processing of the image processing section 101.

The image recognition section 102 inputs the endoscopic image to the trained model 141, allows the trained model 141 to detect the treatment tool from the endoscopic image, and acquires the detection result and the confidence level from the trained model 141. The trained model 141 may further perform image recognition of various tissue or lesions, in addition to the treatment tool. The detection result of the treatment tool, tissue, and lesion may be used for autonomous control of the treatment. The confidence level monitoring section 103 acquires the confidence level of the treatment tool from the image recognition section 102 every frame or at predetermined frame intervals. The lens contamination and fogging determination section 104 compares the confidence level of the treatment tool with the predetermined threshold and, when the confidence level is equal to or smaller than the predetermined threshold, determines that lens contamination or fogging occurs. The control section 106 includes a water supply control section 107. When it is determined that lens contamination or fogging occurs, the water supply control section 107 controls the water supply device 900 to supply water to the objective lens.

The scene recognition section 108 inputs the endoscopic image to the scene recognition trained model 142, allows the scene recognition trained model 142 to detect a treatment scene or a treatment step from the endoscopic image, and acquires the detection result. The scene recognition trained model 142 may be a detector that detects a specific treatment scene or treatment step or may be a classifier that classifies a treatment scene or a treatment step. The control section 106 autonomously controls the endoscope 40 and the treatment tool 50 using a scene detection result. For example, the control section 106 allows the endoscope 40 and the treatment tool 50 to perform a motion programmed in advance in a certain treatment step. When the termination of the treatment step is determined by the scene recognition section 108, the control section 106 proceeds to the next treatment step.

The treatment continuation determination section 105 determines whether to continue the treatment, based on a determination result by the lens contamination and fogging determination section 104, after water supply to the objective lens is performed. In other words, when it is determined that lens contamination or fogging is not eliminated, that is, the confidence level is equal to or smaller than the predetermined threshold, the treatment continuation determination section 105 transmits an instruction to stop the treatment to the control section 106. The control section 106 receives the instruction to stop the treatment and stops the treatment by autonomous control. The image processing section 101 allows the display 300 to display a prompt to manually remove lens contamination when it is determined that lens contamination or fogging is not eliminated.

The control section 106 may perform autonomous control, further using an image recognition result by the image recognition section 102, in addition to the scene detection result. Alternatively, the scene recognition section 108 may input the endoscopic image as well as the image recognition result by the image recognition section 102 to the scene recognition trained model 142, and allow the scene recognition trained model 142 to detect a treatment scene or a treatment step from the endoscopic image and the image recognition result. Alternatively, the scene recognition section 108 and the scene recognition trained model 142 may be omitted, and the control section 106 may perform autonomous control using the image recognition result by the image recognition section 102.

Further, the scene recognition section 108 may input a sensor signal from a sensor provided in the endoscope 40, the treatment tool 50, or the like, and the endoscopic image to the scene recognition trained model 142, and may allow the scene recognition trained model 142 to detect a treatment scene or a treatment step from the sensor signal and the image recognition result. The sensor is, for example, a distance measuring sensor, a force sensor, or a shape detection sensor. The distance measuring sensor is provided, for example, at the distal end of the endoscope 40 to detect three-dimensional information of the field of view. The distance measuring sensor is, for example, a TOF sensor or an ultrasonic distance sensor. The force sensor is provided, for example, at the distal end of the treatment tool 50 to detect contact of the treatment tool 50 with tissue, stress exerted on the treatment tool 50 or the tissue, or the like. An example of the force sensor is a strain gauge. The strain gauge includes a thin insulator and a metal foil resistor on the insulator. Stress exerted on an object having the strain gauge distorts the object as well as the strain gauge, and the distortion changes the resistance of the metal foil resistor. The resistance is measured whereby stress is detected. The shape detection sensor is a sensor that detects the shape of the endoscope or the treatment tool. An example of the shape detection sensor is a UPD or a FBG sensor. The UPD includes coils and an observation device. The coils are provided at predetermined intervals along the longitudinal direction of the endoscope insertion portion or the treatment tool tube. The observation device detects the position of each coil by detecting a magnetic field from the coil and detects the shape of the endoscope or the treatment tool by connecting the position of each coil. The FBG sensor includes an optical fiber and a detection device. The optical fiber is provided along the longitudinal direction of the endoscope insertion portion or the treatment tool tube. Diffraction gratings are formed in the optical fiber. The detection device inputs light to the optical fiber and detects light reflected by the diffraction gratings in the optical fiber. The optical fiber is flexed to conform to the shape of the endoscope or the treatment tool, which changes the wavelength of light reflected from the diffractive gratings. The detection device detects the wavelength, thereby detecting the shape of the endoscope or the treatment tool.

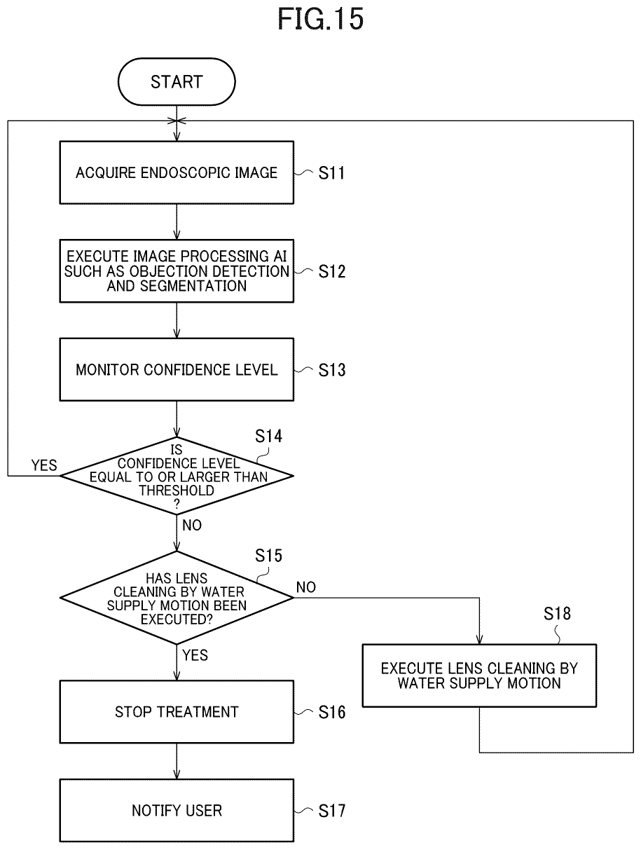

FIG. 15 is a flowchart example illustrating operation of the medical system 1 according to the foregoing first to eighth embodiments.

In step S11, the image processing section 101 acquires an endoscopic image captured by the endoscope 40. In step S12, the image recognition section 102 executes image processing AI such as object detection, segmentation, or key point detection and detects the treatment tool from the endoscopic image. In step S13, the confidence level monitoring section 103 acquires the confidence level from the image processing AI every frame or at predetermined frame intervals. In step S14, the lens contamination and fogging determination section 104 determines whether the confidence level is equal to or larger than the predetermined threshold. If it is determined that the confidence level is equal to or larger than the predetermined threshold, steps S11 to S14 are executed again.

In step S14, if it is determined that the confidence level is not equal to or larger than the predetermined threshold, in step S15, the control section 106 determines whether lens cleaning by the water supply motion has been executed. If lens cleaning has been executed, in step S16, the control section 106 stops the treatment by autonomous control. In step S17, the control section 106 notifies the user to prompt the user to manually remove lens contamination. For example, the control section 106 controls the image processing section 101 to display a prompt to manually remove lens contamination on the display 300. In step S15, if lens cleaning by the water supply motion has not been executed, in step S18, the water supply control section 107 executes lens cleaning by the water supply motion. Subsequently, step S11 and subsequent steps are executed again.

When it is determined that lens contamination or fogging is present, the treatment may be stopped if the field of view is not restored even after cleaning is performed multiple times. In other words, in step S15, the control section 106 may determine whether lens cleaning by the water supply motion has been executed a predetermined number of times. When the predetermined number of times is not reached, the control section 106 may perform lens cleaning again in step S18. When the predetermined number of times is reached, the control section 106 may stop the treatment in step S16.

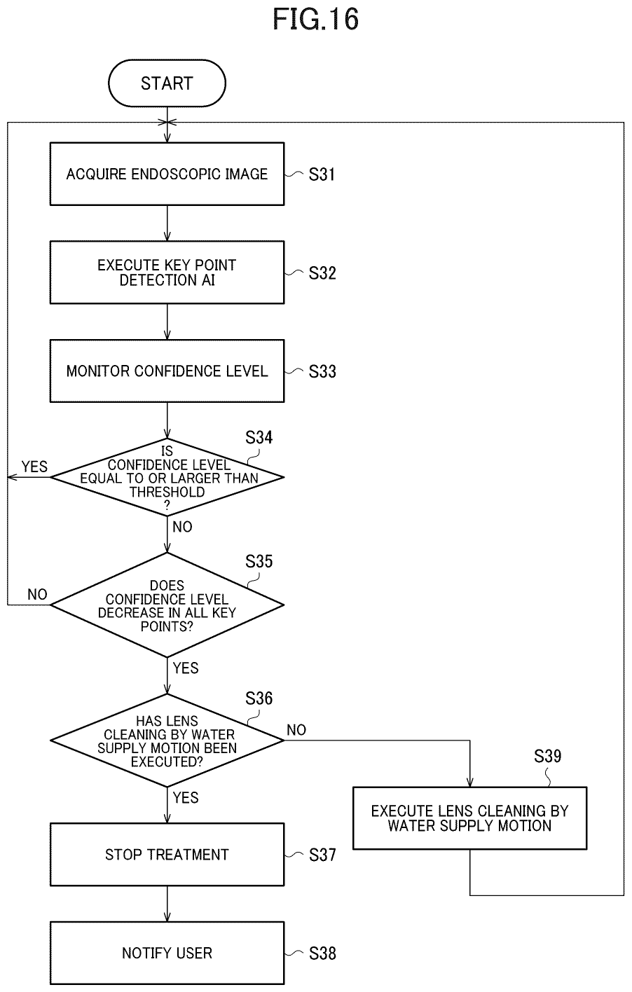

FIG. 16 is a flowchart example illustrating operation of the medical system 1 according to the foregoing ninth embodiment.

In step S31, the image processing section 101 acquires an endoscopic image captured by the endoscope 40. In step S32, the image recognition section 102 executes the key point detection AI and detects a key point of the treatment tool from the endoscopic image. In step S33, the confidence level monitoring section 103 acquires the confidence level from the image processing AI every frame or at predetermined frame intervals. In step S34, the lens contamination and fogging determination section 104 determines whether the confidence level of each key point is equal to or larger than the predetermined threshold. If it is determined that the confidence levels of all of the key points are equal to or larger than the predetermined threshold, steps S31 to S34 are executed again.

In step S34, if it is determined that the confidence levels of one or more key points are not equal to or larger than the predetermined threshold, in step S35, the lens contamination and fogging determination section 104 determines whether the confidence level is equal to or smaller than the predetermined threshold in all of the key points. If there is a key point of which confidence level is not equal to or smaller than the predetermined threshold, steps S31 to S35 are executed again.

In step S35, if the confidence level is equal to or smaller than the predetermined threshold in all of the key points, in step S36, the control section 106 determines whether lens cleaning by the water supply motion has been executed. If lens cleaning has been executed, in step S37, the control section 106 stops the treatment by autonomous control. In step S38, the control section 106 notifies the user to prompt the user to manually remove lens contamination. For example, the control section 106 controls the image processing section 101 to display a prompt to manually remove lens contamination on the display 300. In step S36, if lens cleaning by the water supply motion has not been executed, in step S39, the water supply control section 107 executes lens cleaning by the water supply motion. Subsequently, step S31 and subsequent steps are executed again.

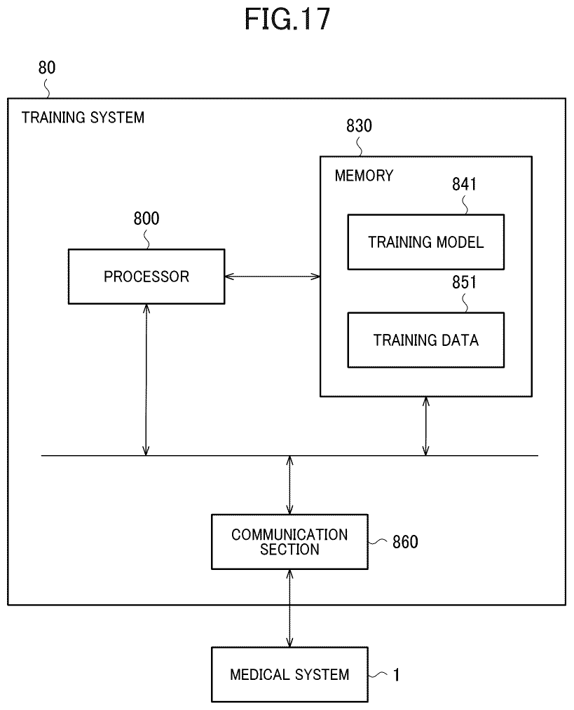

FIG. 17 illustrates a configuration example of a training system 80. The training system 80 includes a processor 800, a memory 830, and a communication section 860.

The processor 800 controls input/output of data between functional sections such as the memory 830 and the communication section 860. The processor 800 is configured with hardware similar to the hardware such as CPU described in conjunction with the processor 100 in FIG. 1. The processor 800 controls data input/output to/from the medical system 1 by executing various computation processing based on a predetermined program read from the memory 830 and an operation input signal and the like from an operation section not illustrated in FIG. 17. The predetermined program here includes a not-illustrated machine learning program. In other words, the processor 800 generates the trained model 141 by reading and executing the machine learning program, necessary data, and the like as appropriate from the memory 830, and executing deep learning processing on a training model 841. The memory 830 stores the not-illustrated machine learning program as well as the training model 841 and training data 851. The memory 830 is configured with hardware such as a semiconductor memory described in conjunction with the memory 130 in FIG. 1.

The communication section 860 is a communication interface that can communicate with the medical system 1 via a predetermined communication scheme. The predetermined communication scheme is, for example, Wi-Fi (registered trademark), but not limited thereto, and may be a communication scheme in conformity with a wired communication standard such as USB. With this configuration, the training system 80 can transmit the trained model 141 trained by machine learning to the medical system 1, and the medical system I can update the trained model 141.

The training system 80 is configured with an information processing device such as a personal computer or a server. Alternatively, the training system 80 may be a cloud system to which a plurality of information processing devices are connected via a network. FIG. 17 illustrates an example in which the training system 80 is separate from the medical system 1, but it is not intended to preclude a configuration example in which the medical system 1 includes a training server corresponding to the training system 80.

The training data 851 includes a plurality of endoscopic images and a ground truth label corresponding to each endoscopic image. The endoscopic images include, for example, endoscopic images that show a variety of treatment tools in a variety of treatment steps. The endoscopic images may include an endoscopic image that does not show a treatment tool. In the object detection AI, the ground truth label includes, for example, a boundary box indicating a position at which each kind of treatment tool is present in an image. In the segmentation, the ground truth label includes, for example, a label having a filled region in which each kind of treatment tool is present in an image. In the key point detection AI, the ground truth label includes, for example, a label indicating a position of each key point. The processor 800 inputs the endoscopic image of the training data 851 to the training model 841, calculates a loss function from the estimation result output by the training model 841 and the ground truth label, and performs feedback to the training model 841 using the loss function to update the training model 841. This is repeated for a large number of endoscopic images to train the training model 841.

The memory 830 may store a scene recognition training model and a scene recognition training data, which are not illustrated in the drawing. The scene recognition training data includes an endoscopic image. The machine learning for the scene recognition training model may be supervised learning or may be unsupervised learning. In the case of supervised learning, the scene recognition training data may further include a ground truth label. The processor 100 generates the scene recognition trained model 142 by training the scene recognition training model so that the scene recognition training model can recognize a treatment scene or a treatment step from the endoscopic image using the scene recognition training data.

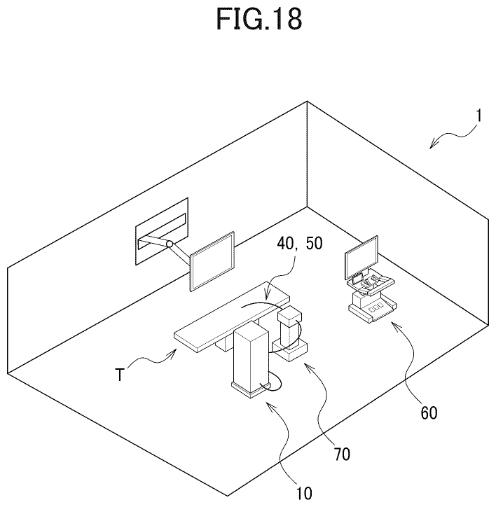

Referring to FIG. 18 to FIG. 25, a more detailed configuration example of the medical system 1 will be described. It is noted that the following detailed configuration example is only by way of example and the medical system 1 may have various configurations as described above.

More specifically, the medical system 1 may be configured as illustrated in FIG. 18. In FIG. 18, the medical system 1 includes the control system 10, the endoscope 40, and the treatment tool 50, and further includes a console 60 and a driving unit 70. The console 60 is connected via wireless communication to the control system 10 using a communication scheme in conformity with a wireless communication standard such as Wi-Fi (registered trademark). The console 60 may be connected via wired communication, for example, via a USB or a LAN. In the medical system 1, the endoscope 40 is inserted into a not-illustrated subject's body lying on a table T to perform various treatments.

The driving unit 70 corresponds to the driving device 20 in FIG. 1 and electrically drives each section of the endoscope 40 and the treatment tool 50 to move based on a control signal from the control system 10. A driving unit of the endoscope 40 and a driving unit of the treatment tool 50 may be provided separately. Further, a driving unit of each of a plurality of treatment tools may be provided separately.

The console 60 is a device that accepts an operation input from a user when the user manually operates the medical system 1. The console 60 includes, for example, a display, a touch panel, a foot pedal, and a handle. The display displays an endoscopic image captured by the endoscope 40 via the control system 10. The touch panel is provided on the display and functions as a pointing device or the like. A specific function is allocated to the foot pedal. When the user presses the foot pedal, the control system 10 executes the specific function. The handle is a device for operating an angle, roll rotation, advancing and retracting, or the like of the endoscope or the treatment tool. When the user operates the handle, the control system 10 allows the endoscope or the treatment tool to perform a motion corresponding to the user operation based on a signal from the handle.

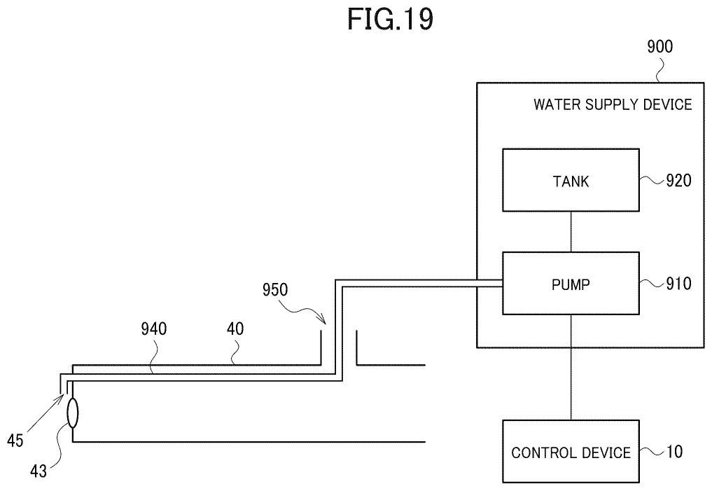

FIG. 19 illustrates a detailed configuration example of the water supply port and the water supply device 900 for washing the objective lens. The endoscope 40 includes a water supply tube 940 and a port 950. The tube 940 is provided along the longitudinal direction of the endoscope insertion portion. The tube 940 has one end connected to the water supply port 45, and the tube 940 has the other end connected to the port 950. The port 950 is provided, for example, at a proximal end of the endoscope insertion portion. The water supply port 45 protrudes from the distal end of the endoscope 40 and is open toward the objective lens 43. The water supply device 900 includes a tank 920 for storing physiological saline, and a pump 910 connected to the tank 920. The pump 910 and the port 950 are connected through a tube. When the control system 10 sends a water supply instruction to the pump 910, the pump 910 feeds the physiological saline in the tank 920 to the tube 940, and the physiological saline is discharged from the water supply port 45 toward the objective lens 43. As a result, the objective lens 43 is cleaned.

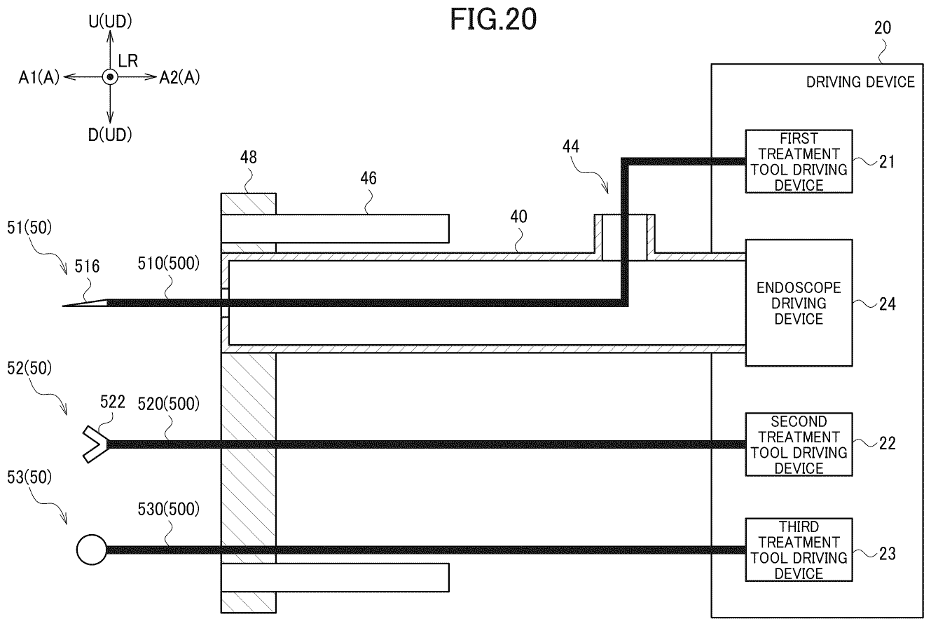

FIG. 20 illustrates a detailed configuration example of the driving device 20. In the example in FIG. 20, a first treatment tool 51, a second treatment tool 52, and a third treatment tool 53 are provided as the treatment tool 50 in FIG. 1. The driving device 20 includes a first treatment tool driving device 21 that controls each portion of the first treatment tool 51, a second treatment tool driving device 22 that controls each portion of the second treatment tool 52, a third treatment tool driving device 23 that controls each portion of the third treatment tool 53, and an endoscope driving device 24 that controls each portion of the endoscope 40. In FIG. 20, some configurations such as the imaging section and the water supply port are not illustrated.

For convenience of explanation, an A axis, a UD axis, and an LR axis are illustrated if necessary as three axes orthogonal to each other, in FIG. 2 and subsequent figures. The A-axis direction is a direction parallel to a direction along the longitudinal direction of a medical manipulator 500 with respect to a distal end portion, which is the distal end of the insertion portion of the endoscope 40. It is assumed that a direction in which the medical manipulator 500 advances is A1 direction, and a direction in which the medical manipulator 500 retracts is A2 direction. In the following description, advancing and retracting may be briefly referred to as “moving forward and backward”. In other words, the A-axis direction is a direction along a direction in which the medical manipulator 500 moves forward and backward. Further, a direction along the UD axis is referred to as UD-axis direction, and a direction along the LR axis is referred to as LR-axis direction.

The first treatment tool 51 is, for example, an injection needle. In this case, the first treatment tool 51 includes a first medical manipulator 510 and an injection needle 516 located at a distal end of the first medical manipulator 510.

The second treatment tool 52 is, for example, grasping forceps. In this case, the second treatment tool 52 includes a second medical manipulator 520 and a grasping portion 522 located at a distal end of the second medical manipulator 520.

The third treatment tool 53 is, for example, a high-frequency knife. In this case, the third treatment tool 53 includes, for example, a third medical manipulator 530. The third treatment tool 53 is configured such that a knife portion can protrude from a distal end of the third medical manipulator 530 as necessary. The knife portion includes a power feed wire, a high-frequency electrode, and the like, which are not illustrated in the drawing. For example, a not-illustrated power feed device feeds high-frequency current to the high-frequency electrode through the power feed wire. In this state, the high-frequency electrode is brought into contact with desired biological tissue so that the biological tissue is ablated by thermal energy generated from the high-frequency electrode. The knife portion illustrated in FIG. 20 has a pole shape. However, the knife portion is not limited to this shape and may be in the shape of a scalpel, a needle, a hook, scissors, or tweezers.

In the example in FIG. 20, the first treatment tool 51, the second treatment tool 52, the third treatment tool 53, and the endoscope 40 are electrically controlled. In other words, the first treatment tool driving device 21 drives the motion of the first treatment tool 51 using an electric actuator, the second treatment tool driving device 22 drives the motion of the second treatment tool 52 using an electric actuator, and the third treatment tool driving device 23 drives the motion of the third treatment tool 53 using an electric actuator. The endoscope driving device 24 drives the motion of the endoscope 40 using an electric actuator. The control system 10 transmits a control signal for controlling an electrically-driven motion to the driving device 20. The first treatment tool driving device 21, the second treatment tool driving device 22, the third treatment tool driving device 23, and the endoscope driving device 24 perform driving based on the control signal, whereby the first treatment tool 51, the second treatment tool 52, the third treatment tool 53, and the endoscope 40 are electrically controlled.

The number of treatment tool channels is not limited to three and may be any number equal to or larger than one. Not all of a plurality of treatment tool channels are provided in the endoscope 40. One or more of the treatment tool channels may be provided outside the endoscope 40. For example, as illustrated in FIG. 20, the medical system 1 may further include an overtube 46. The overtube 46 may have a plurality of treatment tool channels in its inside. For example, the endoscope 40 has one treatment tool insertion opening 44. The first treatment tool 51 is inserted into the treatment tool insertion opening 44, and the second treatment tool 52 and the third treatment tool 53 are respectively inserted into two treatment tool channels in the overtube 46.

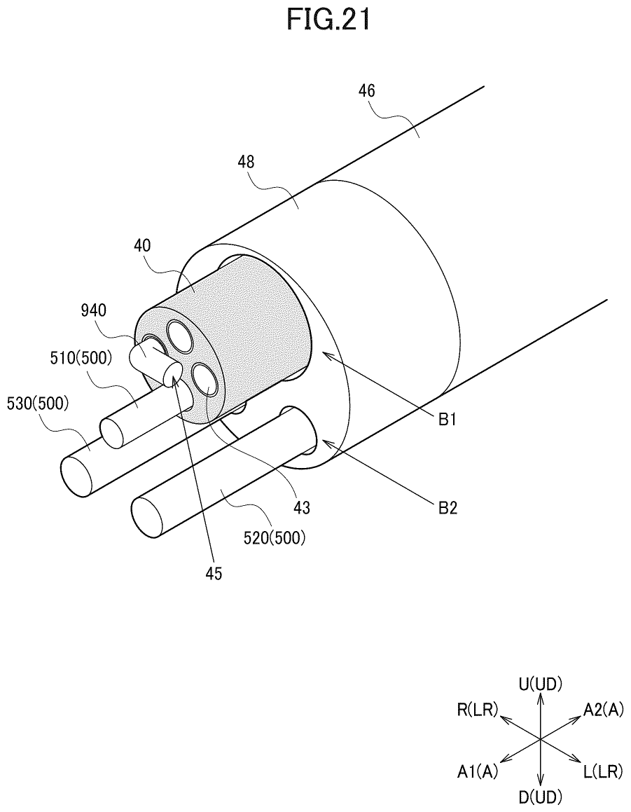

Further, as illustrated in FIG. 20 and FIG. 21, the medical system 1 may further include a cap 48. A variety of known shapes of the cap 48 have been proposed. For example, as illustrated in FIG. 21, the cap 48 has an endoscope outlet hole indicated by B1 and a treatment tool outlet hole indicated by B2. The cap 48 has one endoscope outlet hole and two treatment tool outlet holes. In other words, the first medical manipulator 510 protrudes from the endoscope outlet hole indicated by B1, and the second medical manipulator 520 and the third medical manipulator 530 protrude from the respective treatment tool outlet holes indicated by B2. For convenience of explanation, the distal end of the endoscope 40 is depicted as protruding from the endoscope outlet hole indicated by B1, but the cap 48 may be configured such that the distal end of the endoscope 40 does not protrude. Further, although not depicted in the drawing for the sake of convenience, a tube may be further provided to form a channel between each hole in the cap 48 and the driving device 20.

As illustrated in FIG. 21, the tube 940 for water supply protrudes from the distal end of the endoscope 40 and is bent toward the objective lens 43, and the water supply port 45 which is an opening at one end of the tube 940 faces the objective lens 43. Physiological saline or the like discharged from the water supply port 45 passes through the surface of the objective lens 43 and is then sent into the body.

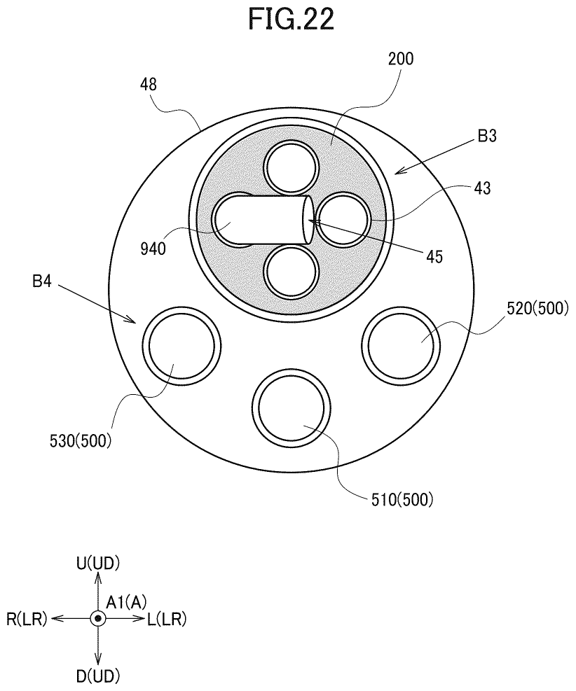

In FIG. 20 and FIG. 21, one treatment tool 50 protrudes from the cap 48 through the insertion portion of the endoscope 40, and two treatment tools 50 protrude from the cap 48 through the overtube 46. However, the configuration is not limited to this. For example, as illustrated in FIG. 22, three treatment tools 50 may protrude from the cap 48 through the overtube 46. The cap 48 in FIG. 22 has one endoscope outlet hole indicated by B3 and three treatment tool outlet holes indicated by B4. Although not illustrated in the drawing, for example, the endoscope 40 may have two treatment tool channels so that two treatment tools 50 may protrude from the cap 48 through the insertion portion of the endoscope 40, and one treatment tool 50 may protrude from the cap 48 through the overtube 46. Further, although not illustrated in the drawing, three treatment tools 50 may protrude from the cap 48 through the insertion portion of the endoscope 40, as an example.

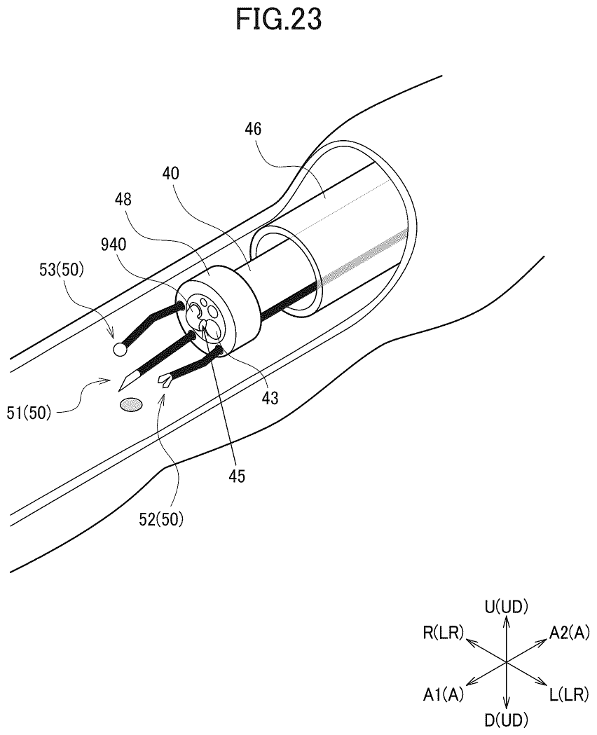

In FIG. 20 and FIG. 21, the cap 48 is fitted on the overtube 46 and integrated with the overtube 46. However, as illustrated in FIG. 23, for example, the cap 48 may be fitted on the endoscope 40 so that the endoscope 40 can be displaced independently of the overtube 46. For example, at the start of a treatment, the endoscope 40, the overtube 46, and the cap 48 may be inserted into the body in an integrated state, and when the distal end portion of the endoscope 40 approaches a site to be treated, the overtube 46 may be secured by a not-illustrated balloon, while the endoscope 40 having the cap 48 fitted thereon may be driven away from the overtube 46.

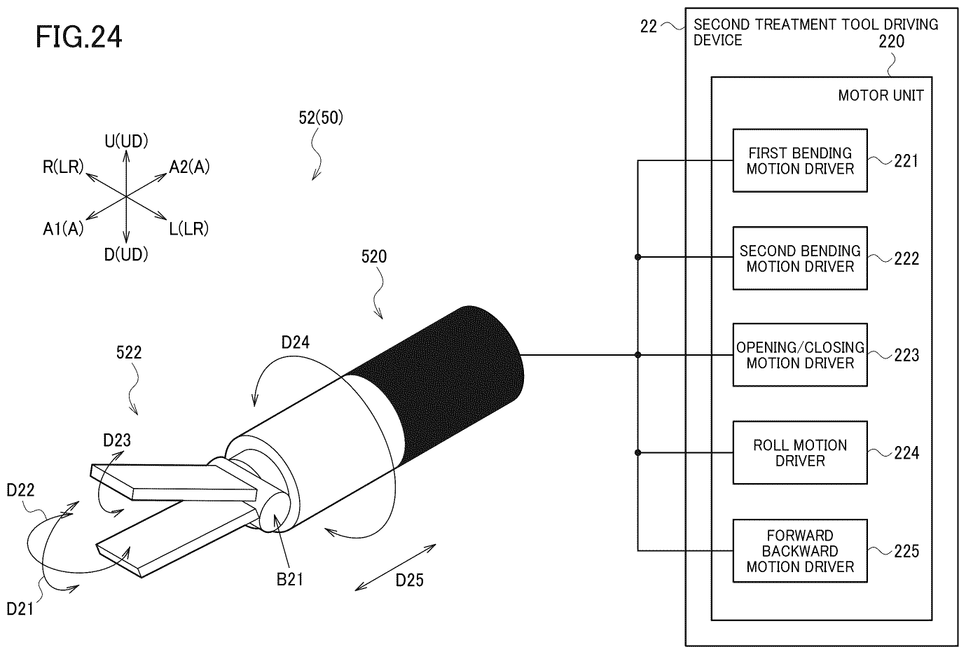

Each of the first treatment tool driving device 21, the second treatment tool driving device 22, the third treatment tool driving device 23, and the endoscope driving device 24 in FIG. 20 includes a drive unit appropriate for the degrees of freedom of the motion of the treatment tool or the endoscope which is a driven target. As an example, a detailed configuration example of the second treatment tool driving device 22 is illustrated in FIG. 24. The second treatment tool driving device 22 includes a motor unit 220. The second treatment tool 52, which is grasping forceps, has the degrees of freedom of a first bending motion, a second bending motion, a forceps opening/closing motion, a roll motion, and a forward-backward motion. The motor unit 220 includes a first bending motion driver 221, a second bending motion driver 222, an opening/closing motion driver 223, a roll motion driver 224, and a forward-backward motion driver 225 corresponding to the above degrees of freedom.

The first bending motion driver 221 pulls or relaxes a not-illustrated pair of wires based on a control signal received from the control system 10 to inflect the second medical manipulator 520 in a direction along the UD axis. As a result, the orientation of the grasping portion 522 is changed along a direction indicated by D21. Similarly, the second bending motion driver 222 inflects the second medical manipulator 520 in a direction along the LR axis based on a control signal received from the control system 10. As a result, the orientation of the grasping portion 522 is changed along a direction indicated by D22.

The opening/closing motion driver 223 controls an opening/closing motion of the grasping portion 522. For example, one of grasping pieces is pivoted around a pivot axis indicated by B21 along a direction indicated by D23, based on a control signal received from the control system 10. The grasping portion 522 in FIG. 24 is illustrated by way of example, and known structures can be widely applied.

The roll motion driver 224 controls a roll rotation motion of a distal end portion of the second medical manipulator 520. For example, the roll motion driver 224 allows the distal end portion of the second medical manipulator 520 to make roll rotation along a direction indicated by D24, based on a control signal received from the control system 10.

The forward-backward motion driver 225 controls a forward-backward motion of the distal end portion of the second medical manipulator 520. The forward-backward motion driver 225 moves the second medical manipulator 520 forward and backward along the A axis, based on a control signal received from the control system 10, for example, using a drive mechanism including a linear motor.

Although not illustrated in the drawing, the first treatment tool driving device 21 can include, for example, a driver similar to the forward-backward motion driver 225, and a plunger driver for injection. The third treatment tool driving device 23 can include, for example, drivers similar to the first bending motion driver 221, the second bending motion driver 222, the roll motion driver 224, and the forward-backward motion driver 225. The endoscope driving device 24 can include, for example, drivers similar to the first bending motion driver 221, the second bending motion driver 222, the roll motion driver 224, and the forward-backward motion driver 225.

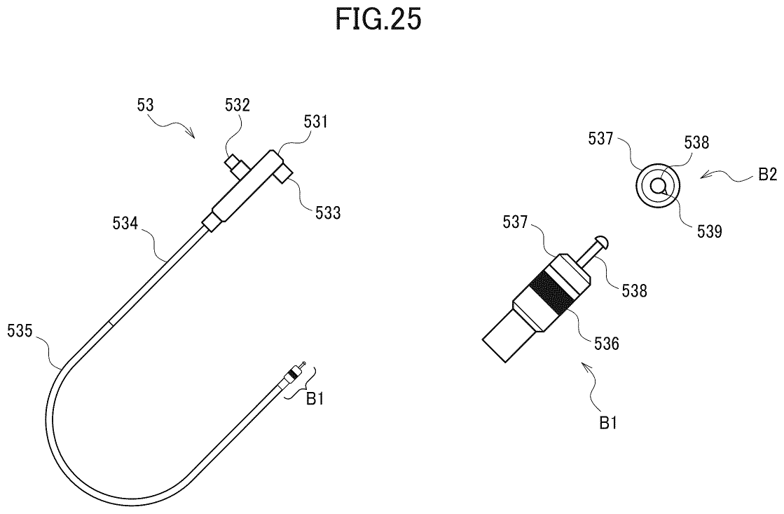

FIG. 25 is a diagram illustrating a detailed configuration example of the third treatment tool 53 which is a high-frequency knife, and a configuration for cleaning the third treatment tool 53.

The third treatment tool 53 includes a proximal end portion 531, a liquid feed port 532, an electrode 533, a connection portion 534, a tube 535, and a distal end portion B1. The proximal end portion 531 is, for example, a cylindrical rigid member. The connection portion 534 is a rigid pipe and has one end connected to the proximal end portion 531. The connection portion 534 has the other end connected to one end of the soft tube 535. The other end of the tube 535 is connected to the distal end portion B1.

The distal end portion B1 includes a distal end cover 537 which is a cylindrical rigid member, a marking 536 attached to a side surface of the distal end cover 537, and a knife 538 protruding from a distal end of the distal end cover 537. B2 is a view of the distal end portion B1 when viewed along the axial direction of the distal end cover 537. As indicated by B2, the distal end cover 537 has a water supply port 539 open in contact with the knife 538.

The electrode 533 is provided, for example, on a side surface of the proximal end portion 531. The electrode 533 is connected to the knife 538 through an electric wire passing through the inside of the proximal end portion 531, the connection portion 534, and the tube 535. The knife 538 is formed of a metal. The electrode 533 is connected to a not-illustrated high frequency generator. High-frequency current output by the high frequency generator is applied from the knife 538 to tissue through the electrode 533 and the electric wire.

The liquid feed port 532 is provided, for example, on the side surface of the proximal end portion 531. The liquid feed port 532 is connected to the water supply port 539 through a liquid feed tube passing through the inside of the proximal end portion 531, the connection portion 534, the tube 535, and the distal end portion B1. The liquid feed port 532 is connected to the water supply device 900. In other words, the water supply device 900 includes a pump for supplying water to the treatment tool, and the pump is connected to the liquid feed port 532 through a tube or the like. Under instructions of the control system 10, the pump feeds physiological saline or the like, so that the physiological saline or the like is discharged from the water supply port 539 through the liquid feed port 532 and the liquid feed tube. As a result, the high-frequency knife 53 is cleaned.

The water supply port 539 may protrude from a side surface of the tube 535 or the distal end cover 537 and may supply water axially forward to the distal end cover 537 and the knife 538. Further, a similar water supply port may be provided in other treatment tools such as the injection needle or the grasping forceps, in addition to the high-frequency knife.

In the present embodiment above, for example, as described in (6) in the first embodiment, the processor 100 may acquire the confidence level again from the trained model 141 after completion of the water supply motion, and may execute the water supply motion again when the confidence level acquired again is equal to or smaller than the predetermined threshold.

According to the present embodiment, when the objective lens is cleaned by the water supply motion but contamination and fogging of the objective lens are not eliminated, the objective lens can be cleaned by performing the water supply motion again.

Further, for example, as described in (6) in the first embodiment, the processor 100 may acquire the confidence level again from the trained model 141 after completion of the water supply motion, and may stop autonomous control when the confidence level acquired again is equal to or smaller than the predetermined threshold.

When the objective lens is cleaned by the water supply motion but contamination and fogging of the objective lens are not eliminated, the accuracy in image recognition such as scene detection is reduced, and consequently the medical system may fail to execute manipulation appropriately. According to the present embodiment, when contamination and fogging of the objective lens are not eliminated, autonomous control of the endoscope and the treatment tool can be stopped.

Further, for example, as described in (5a) in the fourth embodiment, the processor 100 may acquire, from the trained model 141, time-series data of the confidence level corresponding to time-series endoscopic images, and may continue autonomous control when the confidence level is larger than the predetermined threshold over a predetermined period of time after completion of the water supply motion.

Further, for example, as described in (3a) in the second embodiment, the processor 100 may acquire, from the trained model 141, time-series data of the confidence level corresponding to time-series endoscopic images, and may execute the water supply motion when the confidence level is equal to or smaller than the predetermined threshold over a predetermined period of time.

During manipulation, the endoscope and the treatment tool are moved and the endoscopic image changes moment by moment, which may cause noise in the confidence level. According to the present embodiment, the confidence level is monitored over a predetermined period of time, whereby immunity to noise appearing in the confidence level can be improved and lens contamination and fogging can be determined accurately.

Further, for example, as described in (3b) in the third embodiment, the processor 100 may acquire, from the trained model 141, time-series data of the confidence level corresponding to time-series endoscopic images, and may perform smoothing processing on the time-series data of the confidence level. When the confidence level after the smoothing processing is equal to or smaller than the predetermined threshold, the processor 100 may execute the water supply motion.

Further, for example, as described in (5b) in the fifth embodiment, the processor 100 may continue autonomous control when the confidence level after the smoothing processing is larger than the predetermined threshold after completion of the water supply motion.

According to the present embodiment, noise appearing in the confidence level is reduced by performing the smoothing processing on the confidence level. As a result, immunity to noise appearing in the confidence level can be further improved and lens contamination and fogging can be determined more accurately.

Further, for example, as described in (8) in the first embodiment, the processor 100 may obtain a differential value of the confidence level, may estimate, based on the differential value, a scheduled water supply timing when the confidence level becomes equal to or smaller than the predetermined threshold and the water supply motion is to be executed, and may execute the water supply motion before the scheduled water supply timing.

Further, for example, as described in (8a) in the seventh embodiment, the memory 130 may store the scene recognition trained model 142 trained so as to detect a scene of a treatment using the treatment tool 50 from the endoscopic image. The processor 100 may input the endoscopic image to the scene recognition trained model 142 to allow the scene recognition trained model 142 to detect a scene, and may determine a timing to execute the water supply motion, based on the detected scene.

While lens cleaning is performed, the treatment is stopped because the field of view of the endoscope is unable to be obtained. It is therefore desirable to perform lens cleaning at a timing when the efficiency of manipulation is not reduced. According to the present embodiment, when it is expected that the confidence level decreases and the water supply motion is to be performed, lens cleaning is conducted even before the confidence level becomes equal to or smaller than the predetermined threshold. For example, lens cleaning is performed at an appropriate timing such as between treatment steps. This configuration can prevent lens cleaning from interrupting a treatment procedure and can prevent reduction in efficiency of the treatment procedure.

Further, for example, as described in (10) in the ninth embodiment, the processor 100 may acquire, from the trained model 141, a plurality of confidence levels corresponding to a plurality of positions in the endoscopic image, and may continue autonomous control when only one or more confidence levels among the plurality of confidence levels are equal to or smaller than the predetermined threshold.

Further, for example, as described in (10) in the ninth embodiment, the trained model 141 may be trained so as to detect a plurality of key points KYA, KYB, and KYC in the treatment tool 50 from the endoscopic image. The processor 100 may acquire, from the trained model 141, a plurality of confidence levels PKYA, PKYB, and PKYC corresponding to a plurality of key points KYA, KYB, and KYC.

According to the present embodiment, when confidence levels for a plurality of positions in the endoscopic image are obtained, whether to continue the treatment is determined based on a state of the confidence levels. When the confidence level decreases only at one or more of the positions, it can be determined that the treatment tool is contaminated, rather than lens contamination or fogging. This configuration can reduce the possibility of an erroneous determination of lens contamination or fogging and can prevent a treatment stop due to the erroneous determination.

Further, as described in (10) in the ninth embodiment, when only one or more confidence levels among a plurality of confidence levels are equal to or smaller than the predetermined threshold, the processor 100 may execute the water supply motion of washing the treatment tool 50 at a position corresponding to the one or more confidence level.

According to the present embodiment, when it is determined that the treatment tool is contaminated, rather than lens contamination or fogging, the contaminated treatment tool can be cleaned. Cleaning the treatment tool can improve the accuracy of detecting the treatment tool by image recognition. This configuration can improve the accuracy in determining whether lens cleaning is necessary or in autonomous control using image recognition.

Further, for example, as described with reference to FIG. 14, the memory 130 may store the scene recognition trained model 142 trained so as to detect a scene of a treatment using the treatment tool 50 from the endoscopic image. The processor 100 may input the endoscopic image to the scene recognition trained model 142 to allow the scene recognition trained model 142 to detect the scene, and may perform autonomous control using a scene detection result output by the scene recognition trained model 142.

According to the present embodiment, when lens contamination or fogging occurs, the objective lens is cleaned and the field of view is restored. In this case, whether lens cleaning is necessary is determined based on the confidence level of image recognition. This confidence level is an index of the accuracy for AI to recognize an image. When the accuracy is reduced, the accuracy of image recognition is restored. As a result, the accuracy of scene recognition for use in autonomous control is maintained, so that the medical system 1 can continue an appropriate manipulation.

Although the embodiments to which the present disclosure is applied and the modifications thereof have been described in detail above, the present disclosure is not limited to the embodiments and the modifications thereof, and various modifications and variations in components may be made in implementation without departing from the spirit and scope of the present disclosure. The plurality of elements disclosed in the embodiments and the modifications described above may be combined as appropriate to implement the present disclosure in various ways. For example, some of all the elements described in the embodiments and the modifications may be deleted. Furthermore, components in different embodiments and modifications may be combined as appropriate. Thus, various modifications and applications can be made without departing from the spirit and scope of the present disclosure. Any term cited with a different term having a broader meaning or the same meaning at least once in the specification and the drawings can be replaced by the different term in any place in the specification and the drawings.

What is claimed is:

1. A medical system comprising:
an endoscope configured to be electrically driven to move, and to capture an endoscopic image;
a treatment tool configured to be electrically driven to move;
a processor configured to perform autonomous control of electrically-driven motions of the endoscope and the treatment tool and to control a water supply motion; and
a memory configured to store a trained model trained so as to detect the treatment tool from the endoscopic image showing the treatment tool, wherein
the processor inputs the endoscopic image to the trained model to allow the trained model to detect the treatment tool from the endoscopic image, and acquires, from the trained model, a confidence level as to whether a detected target is the treatment tool, and
when the confidence level is equal to or smaller than a predetermined threshold, the processor executes the water supply motion of washing an objective lens of the endoscope.

2. The medical system according to claim 1, wherein
the processor acquires the confidence level again from the trained model after completion of the water supply motion, and
when the confidence level acquired again is equal to or smaller than the predetermined threshold, the processor executes the water supply motion again.

3. The medical system according to claim 1, wherein
the processor acquires the confidence level again from the trained model after completion of the water supply motion, and
when the confidence level acquired again is equal to or smaller than the predetermined threshold, the processor stops the autonomous control.

4. The medical system according to claim 1, wherein
the processor acquires, from the trained model, time-series data of the confidence level corresponding to a time series of the endoscopic image, and
when the confidence level is larger than the predetermined threshold over a predetermined period of time after completion of the water supply motion, the processor continues the autonomous control.

5. The medical system according to claim 1, wherein
the processor acquires, from the trained model, time-series data of the confidence level corresponding to a time series of the endoscopic image, and
when the confidence level is equal to or smaller than the predetermined threshold over a predetermined period of time, the processor executes the water supply motion.

6. The medical system according to claim 1, wherein
the processor acquires, from the trained model, time-series data of the confidence level corresponding to a time series of the endoscopic image, and performs smoothing processing on the time-series data of the confidence level, and
when the confidence level after the smoothing processing is equal to or smaller than the predetermined threshold, the processor executes the water supply motion.

7. The medical system according to claim 6, wherein the processor continues the autonomous control when the confidence level after the smoothing processing is larger than the predetermined threshold, after completion of the water supply motion.

8. The medical system according to claim 1, wherein
the processor
obtains a differential value of the confidence level,
estimates, based on the differential value, a scheduled water supply timing when the confidence level becomes equal to or smaller than the predetermined threshold and the water supply motion is to be executed, and
executes the water supply motion before the scheduled water supply timing.

9. The medical system according to claim 8, wherein
the memory stores a scene recognition trained model trained so as to detect a scene of a treatment using the treatment tool, from the endoscopic image, and
the processor inputs the endoscopic image to the scene recognition trained model to allow the scene recognition trained model to detect the scene, and determines a timing to execute the water supply motion, based on the detected scene.

10. The medical system according to claim 1, wherein
the processor acquires, from the trained model, a plurality of confidence levels corresponding to a plurality of positions in the endoscopic image, and
when only one or more confidence levels among the plurality of confidence levels are equal to or smaller than the predetermined threshold, the processor continues the autonomous control.

11. The medical system according to claim 10, wherein
the trained model is trained so as to detect a plurality of key points in the treatment tool, from the endoscopic image, and
the processor acquires, from the trained model, the plurality of confidence levels corresponding to the plurality of key points.

12. The medical system according to claim 10, wherein when only one or more confidence levels among the plurality of confidence levels are equal to or smaller than the predetermined threshold, the processor executes the water supply motion of washing the treatment tool at a position corresponding to the one or more confidence levels.

13. The medical system according to claim 1, wherein
the memory stores a scene recognition trained model trained so as to detect a scene of a treatment using the treatment tool, from the endoscopic image, and
the processor inputs the endoscopic image to the scene recognition trained model to allow the scene recognition trained model to detect the scene, and performs the autonomous control, using a detection result of the scene output by the scene recognition trained model.

14. A control system comprising:
a processor configured to perform autonomous control of electrically-driven motions of an endoscope and a treatment tool and to control a water supply motion, the endoscope being configured to be electrically driven to move and to capture an endoscopic image, the treatment tool being configured to be electrically driven to move; and
a memory configured to store a trained model trained so as to detect the treatment tool from the endoscopic image showing the treatment tool, wherein
the processor inputs the endoscopic image to the trained model to allow the trained model to detect the treatment tool from the endoscopic image, and acquires, from the trained model, a confidence level as to whether a detected target is the treatment tool, and
when the confidence level is equal to or smaller than a predetermined threshold, the processor executes the water supply motion of washing an objective lens of the endoscope.

15. The control system according to claim 14, wherein
the processor acquires, from the trained model, a plurality of confidence levels corresponding to a plurality of positions in the endoscopic image, and
when only one or more confidence levels among the plurality of confidence levels are equal to or smaller than the predetermined threshold, the processor continues the autonomous control.

16. The control system according to claim 14, wherein
the processor acquires the confidence level again from the trained model after completion of the water supply motion, and
when the confidence level acquired again is equal to or smaller than the predetermined threshold, the processor executes the water supply motion again.

17. The control system according to claim 14, wherein
the processor acquires the confidence level again from the trained model after completion of the water supply motion, and
when the confidence level acquired again is equal to or smaller than the predetermined threshold, the processor stops the autonomous control.

18. A water supply control method comprising:
autonomously controlling electrically-driven motions of an endoscope and a treatment tool, the endoscope being configured to be electrically driven to move and to capture an endoscopic image, the treatment tool being configured to be electrically driven to move;
inputting the endoscopic image to a trained model trained so as to detect the treatment tool from the endoscopic image showing the treatment tool, and allowing the trained model to detect the treatment tool from the endoscopic image;
acquiring, from the trained model, a confidence level as to whether a target detected by the trained model is the treatment tool; and
when the confidence level is equal to or smaller than a predetermined threshold, executing a water supply motion of washing an objective lens of the endoscope.

19. The water supply control method according to claim 18, further comprising:
acquiring, from the trained model, a plurality of confidence levels corresponding to a plurality of positions in the endoscopic image; and
when only one or more confidence levels among the plurality of confidence levels are equal to or smaller than the predetermined threshold, continuing the autonomous control.

20. The water supply control method according to claim 18, further comprising:
acquiring the confidence level again from the trained model after completion of the water supply motion; and
when the confidence level acquired again is equal to or smaller than the predetermined threshold, executing the water supply motion again or stopping the autonomous control.
